(19) United States
(12) Patent Application Publication
(10) Pub. No.: US 2024/0238305 A1
(43) Pub. Date: Jul. 18, 2024

(54) FORMULATIONS OF 3-((3-(4-(2-(ISOBUTYLSULFONYL)PHENOXY)-3-(TRIFLUOROMETHYL)PHENYL)-1,2,4-OXADIAZOL-5-YL)METHYL)-5,5-DIMETHYL-1-(2-MORPHOLINOETHYL)IMIDAZOLIDINE-2,4-DIONE

(71) Applicant: Novartis AG, Basel (CH)

(72) Inventors: Yi FAN, San Diego, CA (US); Declan HARDY, Cambridge, MA (US); Zhihong HUANG, San Diego, CA (US); Jay Parthiban LAKSHMAN, Bridgewater, NJ (US); Jon Christopher LOREN, San Diego, CA (US); Valentina MOLTENI, San Diego, CA (US); Sunil Kumar Mandala RAYABANDLA, Fort Worth, TX (US); Nuria Jimenez RIDRUEJO, Colleyville, TX (US); Duncan SHAW, Boston, MA (US); Jeffrey SMITH, San Diego, CA (US); Christopher Stephen TOWLER, Quincy, MA (US)

(21) Appl. No.: 18/561,444

(22) PCT Filed: May 18, 2022
(86) PCT No.: PCT/IB2022/054640

§ 371 Date: Nov. 16, 2023

Related U.S. Application Data
(60) Provisional application No. 63/191,293, filed on May 20, 2021.
(60) Provisional application No. 63/269,675, filed on Mar. 21, 2022.

(51) Int. Cl. A61K 31/5377; A61K 9/00; A61K 9/10; A61K 47/10; A61K 47/18; A61K 47/26; A61K 47/32; A61K 47/44; A61P 27/04

(52) U.S. Cl. CPC A61K 31/5377; A61K 9/0048; A61K 9/10; A61K 47/10; A61K 47/18; A61K 47/26; A61K 47/32; A61K 47/44; A61P 27/04

(57) ABSTRACT

Formulations of 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione and pharmaceutically acceptable salts thereof, processes for their production, and uses thereof, including in the treatment of ocular diseases and disorders such as dry eye disease and Meibomian gland dysfunction (MGD).

### 1. FIELD OF THE INVENTION

Provided herein are novel formulations of 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione and pharmaceutically acceptable salts thereof, processes for their production, and uses thereof, including in the treatment of ocular diseases and disorders such as Meibomian gland dysfunction (MGD).

### 2. BACKGROUND

Human tear film is comprised of three layers. The mucus layer coats the cornea forming a foundation so the tear film can adhere to the eye. The middle aqueous layer provides moisture and supplies oxygen and other important nutrients to the cornea. The outer lipid layer is an oily film that seals the tear film on the eye and helps to prevent evaporation of the layers beneath.

A large part of the lipids that contribute to the tear film are made in the meibomian gland, which is a holocrine type of exocrine gland, at the rim of the eyelid inside the tarsal plate. Meibomian glands are primarily responsible for lipid generation, and abnormal lipid secretions from in these glands can affect the various functions required of the tear film. For instance, the lipid layer of tear film prevents evaporation, lowers surface tension of tears thereby preventing spillover of tears from the lid margin and tear film lipids also play a role in the ability of the tear film to spread on the ocular surface and thereby influences the interaction between the lid and ocular surface to prevent damage to either surface. All these properties of the tear film are influenced by the lipids provided by the meibomian gland to the ocular surface to be incorporated into the tear film,

Dysfunction of the meibomian glands can lead to lipid insufficiency that destabilizes the tear film and causes decreases in tear film break-up time and evaporative dry eye (see, e.g., Sullivan et al., Ann. NY Acad. Sci., 966, 211-222, 2002). Meibomian gland dysfunction (MGD), also known as meibomitis, posterior blepharitis or inflammation of the meibomian glands, is a chronic, diffuse abnormality of the meibomian glands, commonly characterized by terminal duct obstruction and/or qualitative/quantitative changes in the glandular secretion (Nelson J D, et al., Invest Ophthalmol Vis Sci 2011; 52:1930-7). MGD is further characterized by meibum with higher viscosity and melting temperature, as well as a suboptimal tear lipid layer that cannot prevent the evaporation of tears.

[symbol]

alteration of the tear film, symptoms of eye irritation, clinically apparent inflammation, and ocular surface disease. MGD often causes dry eye, and may contribute to blepharitis. There is high unmet medical need with symptomatic MGD in ˜3.5% of population presenting in up to ˜70% of evaporative dry eye disease patients.

MGD may also be characterized by increased melting point of the lipids, causing solidification of the lipids and obstruction of the meibomian gland secretion. This can result in cysts, infections and decreased lipid content in the tears.

Commonly used methods to treat Meibomian gland dysfunction include warm compresses to eyelid margins or mechanical treatments that apply heat and pressure to express the glands (eg, LipiFlow) or even mechanical probing of meibomian ducts. Other treatments include infrared devices to provide intense pulsed light (IPL) treatments or chemicals to eyelid margins to induce tear lipid melting and secretion. For inflammation, glucocorticoids may be used and antibiotics like penicillin, doxycycline, and tetracyclines may be used, although neither glucocorticoids nor antibiotics have been approved for this use by the FDA. Additionally, these therapies are not suitable for long term use, either for side-effects or for a lack of demonstrated efficacy. There is a long-felt and unmet need for safe, effective treatments for the treatment of Meibomian gland dysfunction that can improve lipid quality and tear film.

The liver X receptors (LXRs) are ligand-activated transcription factors of the nuclear receptor superfamily and were first described by Willy, P. J., et al., “LXR, a nuclear receptor that defines a distinct retinoid response pathway,” Genes & Development 9:1033-1045 (Cold Spring Harbor Laboratory Press). LXRs comprise two isoforms (LXR alpha and LXR beta) which are highly expressed in the epidermis. LXR transcriptionally regulates a number of processes involved in lipid homestasis including cholesterol transport and fatty acid synthesis. For example, it has been reported that stearoyl-coenzyme A desaturase 1 (SCD1), the enzyme necessary for the biosynthesis of monounsaturated fatty acids, is a direct transcriptional target of LXR. See Shultz et al., Genes & Dev. 2000. 14: 2831-2838. Other biological pathways that are regulated by these actions of LXR include the stimulation of epidermal lipid synthesis, which can increase lamellar body formation, secretion and processing in the stratum corneum, which leads to formation of lamellar membranes that regulate the permeability of the corneal barrier. LXR also has a complex interaction with inflammatory pathways. While LXR can transcriptionally downregulate a number of inflammatory cytokines, it can also be itself downregulated by inflammatory pathways. The ability of LXR to reduce inflammation has been the explored in indications such as atherosclerosis in which macrophages play a role in the pathology.

Overall, the current treatments for Meibomian gland dysfunction do not adequately address pharmacologically the cause or pathology of disease and there remains a need for new treatments or therapies.

### 3. SUMMARY

3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione (“Compound 1”) is an LXR agonist having biological activity that makes the compound useful for treating ocular diseases such as MGD (see, e.g., Example 3). However, its physical properties make formulating Compound 1 for therapeutic use (e.g., in human subjects) difficult. Compound 1 is poorly water soluble, making it difficult to formulate compositions of Compound 1 with desired concentrations of Compound 1 for therapeutic use. For example, Compound 1 was found to have a solubility in 10 mM acetate, pH 4 of only 0.025 mg/ml and a solubility in 10 mM acetate, pH 5 of only 0.001 mg/ml. In a 10 mM phosphate buffer at pH 7 (a physiological pH), the solubility of Compound 1 was found to be below the limit of quantification. Moreover, Compound 1 has a melting temperature of only 118° C., which makes heat sterilization of Compound 1 difficult. In spite of these challenges, formulations of Compound 1 which can be used, for example, in the treatment of ocular diseases and disorders such as dry eye disease and MGD, have been developed.

Accordingly, in various aspects, the present invention provides formulations of Compound 1 and pharmaceutically acceptable salts thereof, processes for their production, kits comprising formulations of Compound 1, and methods for using the formulations and kits, for example in the treatment of ocular diseases and disorders such as dry eye disease and Meibomian gland dysfunction (MGD).

In some aspects, the invention provides formulations comprising Compound 1 or a pharmaceutically acceptable salt thereof and one or more pharmaceutically acceptable carriers, for example one or more castor oil-based solubilizers. In some embodiments, the formulations are aqueous suspensions intended for topical administration to a subject's eyelids (e.g., for administration by the subject's finger or by an applicator to the eyelids). Exemplary features of formulations of the invention and components thereof are described in Section 5.2 and specific embodiments 1 to 203 and 229, infra.

In other aspects, the invention provides kits comprising a formulation described herein and a container, for example, a droptainer for single or multiple use. Exemplary features of kits and components thereof are described in Section 5.2 and specific embodiments 204 to 208, infra.

In other aspects, the invention provides processes of making formulations and kits described herein. Exemplary processes are described in Section 5.3 and specific embodiments 209 to 228, infra.

In further aspects, the invention provides methods of agonizing LXR in the meibomian glands of a subject, methods of increasing the ratio of desaturated lipids to saturated lipids in the eye a subject, methods of lowering a subject's meibum melting temperature and/or increasing meibum outflow from a subject's meibomian glands, and methods of reducing obstruction of meibum outflow from a subject's meibomian glands, the methods comprising administering an amount of a formulation of Compound 1 described herein to the subject, for example, to the subject's eyelids (e.g., for administration by the subject's finger or by an applicator to the eyelids). Exemplary features of such methods are described in Section 5.4 and specific embodiments 230 to 237, infra.

In yet further aspects, the invention provides methods for treating dry eye (e.g., dry eye associated with Meibomian gland dysfunction (MGD)) and methods of treating Meibomian gland dysfunction (MGD) in a subject in need thereof comprising administering a therapeutically effective amount of a formulation of Compound 1 described herein to the subject, for example to the subject's eyelids (e.g., for administration by the subject's finger or by an applicator to the eyelids). Exemplary features of such methods are described in Section 5.4 and specific embodiments 238 to 244, infra.

In still further aspects, the invention provides formulations of Compound 1 described herein for use in methods of agonizing LXR in the meibomian glands of a subject, methods of increasing the ratio of desaturated lipids to saturated lipids in the eye a subject, methods of lowering a subject's meibum melting temperature and/or increasing meibum outflow from a subject's meibomian glands, methods of reducing obstruction of meibum outflow from a subject's meibomian glands, methods of treating dry eye disease (e.g., associated with MGD), and methods of treating MGD. Exemplary features of such formulations for use are described in Section 5.4 and specific embodiments 245 to 253, infra.

In still further aspects, the invention provides irradiated Compound 1 and pharmaceutically acceptable salts thereof. Irradiated Compound 1 and pharmaceutically acceptable salts thereof can be used, for example, in the manufacture of a medicament, e.g., for treating a disease or condition described herein, or in the manufacture of a formulation described herein. Exemplary features of irradiated Compound 1 and pharmaceutically acceptable salt thereof and uses thereof are described in Sections 5.2 to 5.4 and specific embodiments 254 to 258, infra.

### 4. BRIEF DESCRIPTION OF THE FIGURES

FIG. 1 provides an illustrative X-ray powder diffraction pattern (XRPD) of the crystalline form of Compound 1, designated herein as Form A, showing degrees 26 (2-theta) on the X-axis and intensity on the Y-axis. More detailed listings of the XRPD peaks for Form A are set forth in Table 1.

FIG. 2 provides an illustrative differential scanning calorimetry (DSC) profile of the crystalline form of Compound 1, designated herein as Form A. The DSC graph plots the heat flow as a function of temperature from a sample, the temperature rate change being about 10 K/min.

FIG. 3 provides an illustrative thermogravimetric analysis (TGA) profile of the crystalline form of Compound 1, designated herein as Form A. The TGA profile graphs the percent loss of weight of the sample as a function of temperature, the temperature rate change being about 10 K/min.

FIG. 4 demonstrates the decrease in meibum melting temperature measured upon administration of Compound 1 to rat eyes at a concentration of 1%.

FIGS. 5A-5B show various formulations of Compound 1. FIG. 5A: formulations that do not show noticeable settling over 7 or 14 days; FIG. 5B: formulations that show noticeable settling over 14 days.

FIGS. 6A-6C show rabbit meibomian gland exposure to Compound 1 following topical administration of various Compound 1 formulations FIG. 6A: area under the curve (AUC) from 0 to 12 hours from administration; FIG. 6B: Compound 1 concentration at 12 hours post administration; FIG. 6C: plot of Compound 1 concentrations measured at 0.5, 6, and 12 hours post administration. Upper and lower meibomian glands from both eyes of animals combined for N=8.

### 5. DETAILED DESCRIPTION

### 5.1. Definitions

“A,” “an,” “the” and similar terms as used herein (especially in the context of the claims) are to be construed to cover both the singular and plural unless otherwise indicated herein or clearly contradicted by the context.

The term “carbomer” as used herein refers to synthetic high-molecular-weight polymers of acrylic acid that are crosslinked, e.g., with allyl sucrose or allyl ethers of pentaerythritol. The polymers are often characterized as having carboxylic acid functional groups and preferably contain from 2 to 7 carbon atoms per functional group. Carbomers are available under the trade name CARBOPOL® from various suppl

[symbol]

ar embodiments, the carbomer is carbomer homopolymer Type B. In particular embodiments, the carbomer is CARBOPOL® 934P (Carbomer 934P), 940, 941 or 974P. In particular embodiments, the carbomer is CARBOPOL® 974P (Carbomer 974P).

The term “castor oil-based solubilizer” as used herein refers to a compound or mixture of compounds comprising castor oil and/or derived from castor oil. For example, castor oil-based solubilizers include solubilizers produced by reacting castor oil with ethylene oxide, or reacting hydrogenated castor oil with ethylene oxide. Exemplary castor oil-based solubilizers include polyoxyl 40 hydrogenated castor oil (marketed by BASF as KOLLIPHOR® RH40) and polyoxyl 35 castor oil (marketed by BASF as KOLLIPHOR® EL).

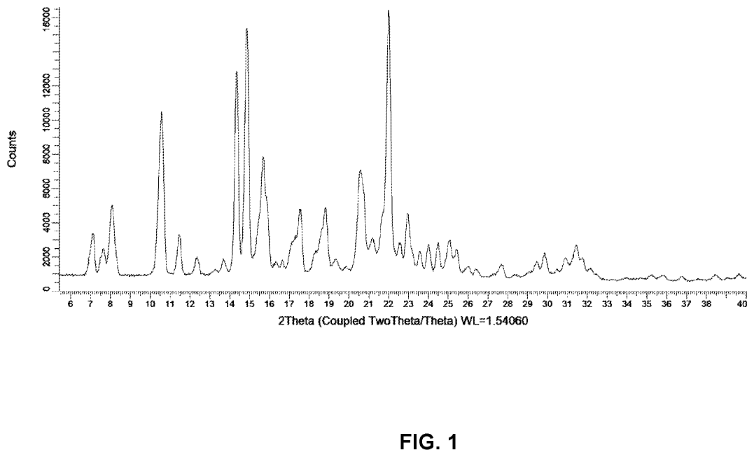

The term “Compound 1” as used herein refers to 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione. In some embodiments, Compound 1 is in “Form A.” In some embodiments, Form A is characterized by an XRPD pattern comprising one or more peaks selected from 7.2, 8.2, 10.7, 14.5, 15.0, 20.7, 21.8±0.2° 2θ. In some embodiments, Form A is characterized by an XRPD pattern comprising two, three, or four representative peaks selected from 7.2, 8.2, 10.7, 14.5, 15.0, 20.7, 21.8±0.2° 2θ. In other embodiments, Form A is characterized by an XRPD pattern comprising one or more peaks selected from FIG. 1, as shown in Table 1.

The term “D90 particle size” as used herein refers to the diameter value in a volumetric particle size distribution for which 90% of the particles (by volume) have a diameter at or below. Thus, for example, for a composition having a D90 particle size of 1 μm, 90% of the particles by volume have a diameter of 1 μm or less. The D90 particle size can be measured by laser diffraction. Exemplary laser diffraction particle size analyzers that can be used to measure D90 particle size include the Microtrac S3500, Sync, and Bluewave particle size analyzers.

The term “D50 particle size” as used herein refers to the diameter value in a volumetric particle size distribution for which 50% of the particles (by volume) have a diameter at or below. Thus, for example, for a composition having a D50 particle size of 1 μm, 50% of the particles by volume have a diameter of 1 μm or less. The D50 particle size can be measured by laser diffraction. Exemplary laser diffraction particle size analyzers that can be used to measure D50 particle size include the Microtrac S3500, Sync, and Bluewave particle size analyzers.

The term “D10 particle size” as used herein refers to the diameter value in a volumetric particle size distribution for which 10% of the particles (by volume) have a diameter at or below. Thus, for example, for a composition having a D10 particle size of 1 μm, 10% of the particles by volume have a diameter of 1 μm or less. The D10 particle size can be measured by laser diffraction. Exemplary laser diffraction particle size analyzers that can be used to measure D10 particle size include the Microtrac S3500, Sync, and Bluewave particle size analyzers.

The term “LiverX receptor” or “LXR” as used herein refers to a nuclear receptor implicated in cholesterol biosynthesis. As used herein, the term LXR refers to both LXRα and LXRβ isoforms of the protein found in mammals and fragments thereof. Exemplary LXR protein sequences include UniProt identifier Q13133 (exemplary human LXRα protein sequence) and UniProt identifier P55055 (exemplary human LXRβ protein sequence).

The term “humectant” as used herein refers to a substance suitable for application to skin and which promotes retention of water in a formulation containing the humectant and/or skin to which the formulation is applied. Exemplary humectants include glycerin, propylene glycol, mannitol, and sorbitol.

The term “ophthalmically compatible”, as used herein refers to formulations, polymers and other materials and/or dosage forms which are suitable for use in contact with the ocular tissues of subject, including human beings and animals, without excessive toxicity, irritation, allergic response, or other problem or complication, commensurate with a reasonable benefit/risk ratio.

The term “pharmaceutically acceptable carrier”, as used herein, refers to a substance useful in the preparation or use of a formulation and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, humectants, sweetening agents, flavoring agents, dyes, and combinations thereof, as would be known to those skilled in the art (see, for example, Remington The Science and Practice of Pharmacy, 22^{nd} Ed. Pharmaceutical Press, 2013, pp. 1049-1070).

The term “preservative” as used herein, refers to a substance for prolonging shelf-life and/or inhibiting the growth of microorganisms (e.g., bacteria, fungi, viruses, and protozoa) in a formulation. Exemplary preservatives include quaternary ammonium compounds such as benzalkonium chloride, benzoxonium chloride (e.g., N-benzyl-N—(C8-C18 dimethylammonium chloride) and the like. Examples of preservatives different from quaternary ammonium salts include, e.g., alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenylethanol, guanidine derivatives, such as, for example, chlorohexidine or polyhexamethylene biguanide, sodium perborate, or sorbic acid. Where appropriate, a sufficient amount of preservative may be added to a formulation to protect against secondary-contaminations during use, e.g., caused by bacteria and/or fungi. In some embodiments, the formulations provided herein do not comprise a preservative. In some embodiments, the formulations provided here comprise a preservative.

The term “prevent”, “preventing” or “prevention” of any disease or disorder as used herein refers to the prophylactic treatment of the disease or disorder; or delaying the onset or progression of the disease or disorder.

The terms, “salt” or “salts” as used herein refers to an acid addition or base addition salt of a compound provided herein. “Salts” include in particular “pharmaceutically acceptable salts”. “Pharmaceutically acceptable salts” as used herein refers to salts that retain the biological effectiveness and properties of Compound 1 and, which typically are not biologically or otherwise undesirable. The skilled artisan will appreciate that salts, including pharmaceutically acceptable salts, of Compound 1 may be prepared. These salts may be prepared in situ during the final isolation and purification of the compound, or by separately reacting the purified compound in its free form with a suitable base or acid.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases, such as carboxylate, sulfonate and phosphate salts.

The term “stable” as used herein with reference to a parameter of a formulation (e.g., viscosity, osmolality, pH, resuspendability, etc.) means that the parameter does not change to an unacceptable degree during a defined period of storage, for example for a period of 6 weeks, 3 months, or 6 months, for example at 5° C., 25° C., or 40° C. For example, if a formulation has a specification for a given parameter, the parameter can be considered stable if the parameter is within the specification when measured before the period of storage and when measured after the period of storage. For example, if the specification for viscosity for a formulation is 200 cPs to 400 cPs, the viscosity can be considered stable for a defined period of storage if the measured viscosity is within the range of 200 cPs to 400 cPs before the storage and after the storage.

The term “subject” as used herein refers to a living organism suffering from one or more of the diseases or disorders described here that can be treated by administration of a formulation described herein. Examples of subjects include mammals (e.g., humans and animals such as dogs, cows, horses, monkeys, pigs, guinea pigs, sheep, goats, cats, mice, rabbits, rats, and transgenic non-human animals). In certain embodiments, the subject is a primate. In certain embodiments, the subject is a human, e.g., a human suffering from, at risk of suffering from, or potentially capable of suffering from a disease described herein. In particular embodiments, the subject is an adult human at least 18 years of age. In particular embodiments, the subject is an adult human from 18 to 75 years of age. In some embodiments, the subject is a human child up to 18 years of age.

The term “a subject is in need of a treatment” refers to if such subject would benefit biologically, medically or in quality of life from such treatment.

The term “substantially pure”, as used herein, when used in reference to a form, such as Form A, means a compound having a purity greater than 90 weight %, including greater than 90, 91, 92, 93, 94, 95, 96, 97, 98, and 99 weight %, and also including equal to 100 weight % of 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione, based on the weight of the compound. The remaining material comprises other form(s) of the compound, and/or reaction impurities and/or processing impurities arising from its preparation. For example, a crystalline form of 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione may be deemed substantially pure in that it has a purity greater than 90 weight %, as measured by means that are at this time known and generally accepted in the art, where the remaining less than 10 weight % of material comprises other form(s) of 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione and/or reaction impurities and/or processing impurities.

The term “substantially the same”, as used herein with reference to X-ray diffraction peak positions means that typical peak position and intensity variability are taken into account. For example, one skilled in the art will appreciate that the peak positions (°2θ) will show some inter-apparatus variability, typically as much as ±0.2° 2θ. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measure only.

The term “therapeutically effective amount”, as used herein refers to an amount of a formulation of Compound 1 that will elicit the biological or medical response of a subject, for example, increase enzyme or a protein activity, or ameliorate symptoms, alleviate conditions, slow or delay disease progression, or prevent a disease, etc. In one embodiment, the term “a therapeutically effective amount” refers to the amount of a formulation that, when administered to a subject, is effective to agonize LXR and thereby at least partially alleviate, prevent and/or ameliorate Meibomian gland dysfunction. In another embodiment, the term “a therapeutically effective amount” refers to the amount of formulation provided herein that, when administered to a cell, or a tissue, or a non-cellular biological material, or a medium, is effective to increase the activity of LXR.

The terms “treat”, “treating” or “treatment” of any disease or disorder, as used herein, refers to relieve, alleviate, delay, reduce, reverse, or improve at least one symptom or sign of a condition in a subject. In one embodiment, the term “treating” refers to relieving, alleviating, delaying, reducing, reversing, or improving at least one symptom or sign selected from abnormal meibomian gland secretions, Meibomian gland dysfunction, dry eye, meibomian gland secretions, redness of the eyelid margins, burning and/or itching in a subject's eye, ocular discomfort, corneal epithelial erosion, ocular and conjunctival staining, and reducing blurred and/or fuzzy vision. The term “treating” may also mean to arrest, delay the onset (i.e., the period prior to clinical manifestation of a disease) and/or reduce the risk of developing or worsening a condition.

The term “viscosity” as used herein, unless otherwise defined in a particular context, refers to viscosity of a composition (e.g., a formulation described herein) measured by a viscometer at 25° C. In some embodiments, viscosity is measured using a Brookfield viscometer with spindle CP-52 at 10 rpm at 25° C.

The term “3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities” as used herein refers to impurities in a formulation attributable to synthesis and/or degradation of Compound 1 or a pharmaceutically acceptable salt thereof. High pressure liquid chromatography (HPLC) can be used to quantify 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities.

An increase in the amount of 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities can be reported following a period of storage. For example, if the amount of 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities prior to a period of storage is 0.8% (where the sum of the amounts of Compound 1 or a pharmaceutically acceptable salt thereof and all 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities totals 100%), and 1.2% following a period of storage, the increase in 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities can be reported as 0.4%.

Unless indicated otherwise, all component concentrations are presented in units of % weight/weight (% w/w). As is commonly understood, the % w/w value refers to the proportion of a particular component within a formulation, as measured by weight of the component to the total weight of the formulation.

### 5.2. Compound 1, Formulations Thereof, Formulation Components, and Kits

In one aspect, the invention provides a formulation comprising Compound 1 or a pharmaceutically acceptable salt thereof and one or more pharmaceutically acceptable carriers. Pharmaceutically acceptable carriers include, for example, solubilizers, viscosity enhancers, tonicity enhancers, humectants, and pH adjusters. Exemplary features of Compound 1 and pharmaceutically acceptable salts are described in Section 5.2.1, and exemplary pharmaceutically acceptable carriers that can be included in the formulations of the invention are described in Section 5.2.2 (including its subparts). In some embodiments, the formulation can be self-preserved and does not include a preservative. In some embodiments, the formulation further comprises a preservative.

The formulations of the invention are typically aqueous suspensions. A suspension is a heterogeneous mixture of a fluid that contains solid particles sufficiently large for sedimentation. The particles in a suspension can be characterized by their size distribution, for example by D90, D50 and D10 values. For ophthalmic formulations, for example formulations intended for topical administration to a subject's eyelid (e.g., by spreading the formulation over the eyelid surface, for example with the subject's finger or with an applicator), smaller particle sizes are desirable in order to avoid or limit eye irritation caused by large particles. Thus, in various embodiments, the aqueous suspensions of the invention have a D90 particle size of no more than 20 μm, no more than 15 μm, no more than 10 μm, no more than 5 μm, no more than 3 μm, no more than 2 μm, or no more than 1 μm. In some embodiments, the D90 particle size is at least 0.5 μm or at least 6 μm.

D50 particle size for a given formulation is smaller than its D90 particle size, and in some embodiments, D50 particle size of the aqueous suspensions of the invention is no more than 5 μm, no more than 3 μm, no more than 2 μm, no more than 1 μm, no more than 0.9 μm, no more than 0.8 μm, no more than 0.7 μm, no more than 0.6 μm, no more than 0.5 μm, no more than 0.4 μm, or no more than 0.3 μm. In some embodiments, D50 particle size is at least 0.2 μm, at least 0.3 μm, at least 0.4 μm, at least 0.5 μm, at least 0.6 μm, or at least 0.7 μm.

D10 particle size for a given formulation is smaller than its D50 particle size, and in some embodiments, the D10 particle size of the aqueous suspensions of the invention is no more than 2 μm, no more than 1 μm, no more than 0.9 μm, no more than 0.8 μm, no more than 0.7 μm, no more than 0.6 μm, no more than 0.5 μm, no more than 0.4 μm, or no more than 0.3 μm. In some embodiments, the D10 particle size is at least 0.1 μm, at least 0.2 μm, at least 0.3 μm, or at least 0.1 μm.

Particle size of Compound 1 or a pharmaceutically acceptable salt thereof as reflected by D90 and/or D50 and or D10 particle size, can be reduced, for example, by wet milling Compound 1 or the pharmaceutically acceptable salt thereof together with one or more components of a final formulation, e.g., a solubilizer such as a castor oil-based solubilizer. The formulations of the invention typically comprise 0.1% w/w to 5% w/w of Compound 1 or a pharmaceutically acceptable salt thereof (e.g., at least 0.1% w/w, at least 0.3% w/w, at least 0.5% w/w, at least 1% w/w, at least 2% w/w, or at least 3% w/w to no more than 5% w/w, no more than 4% w/w, no more than 3% w/w, no more than 2% w/w or no more than 1% w/w). In some embodiments, formulations of the invention comprise 0.1% w/w, 0.2% w/w, 0.3% w/w, 0.4% w/w, 0.5% w/w, 1% w/w, 2% w/w, 3% w/t, or 5% w/w of Compound 1 or a pharmaceutically acceptable salt thereof. Unless otherwise specified, the weight or dosage referred to herein for Compound 1 or a salt thereof provided herein is the weight or dosage of the compound itself, not that of the salt thereof, which can be different to achieve an intended therapeutic effect. For example, the weight or dosage of a corresponding salt of Compound 1 suitable for the methods or compositions disclosed herein may be calculated based on the ratio of the molecular weights of the salt and compound itself. In some embodiments, formulations of the invention comprise Compound 1 in free (non-salt) form.

Formulations of the invention are preferably stable during periods of storage. Various parameters can be measured to assess stability, for example, amount of total impurities (e.g., due to degradation of Compound 1), particle size diameter (e.g., D90, D50, D10), viscosity, osmolality, pH, resuspendability, and appearance. In some embodiments, formulations of the invention exhibit stability for one, two, three, four, five, six or seven of the following parameters when stored for 6 weeks, 3 months, or 6 months at 5° C., 25° C., or 40° C.: i) 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities; ii) D90 particle size; iii) viscosity; iv) osmolality; v) pH; vi) resuspendability; vii) appearance.

In some embodiments, a formulation can be considered to have stability with respect to 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities for a given storage condition when the amount of impurities increases by no more than 1% or 0.5% relative to the amount of impurities present prior to storage.

In some embodiments, a formulation can be considered to have stability with respect to D90 particle size for a given storage condition when the D90 particle size increases no more than 500%, 100%, 50%, or 10% relative to the D90 particle size prior to storage.

In some embodiments, a formulation can be considered to have stability with respect to viscosity for a given storage condition when the viscosity at the end of storage is 70% to 150%, 80% to 125%, or 90% to 110% of the measured viscosity prior to storage.

In some embodiments, a formulation can be considered to have stability with respect to osmolality for a given storage condition when the osmolality at the end of storage is 90% to 120% or 95% to 110% of the measured osmolality prior to storage.

In some embodiments, a formulation can be considered to have stability with respect to pH for a given storage condition when the pH at the end of storage is not more than 0.1, 0.2, or 0.3 pH units above or below the pH of the formulation prior to storage.

In some embodiments, a formulation can be considered to have stability with respect to resuspendability for a given storage condition when the resuspendability at the end of storage is qualitatively the same as the resuspendability of the formulation prior to storage. For example a formulation that by visual inspection appears to be homogeneous and free of lumps before and after storage can be considered to have stability with respect to resuspendability.

In some embodiments, a formulation can be considered to have stability with respect to appearance for a given storage condition when the appearance at the end of storage is qualitatively the same as the appearance of the formulation prior to storage. For example, a formulation that by visual inspection appears to be the same color before and after storage can be considered to have stability with respect to appearance.

Formulations of the invention that are aqueous suspensions can in some embodiments exhibit noticeable settling by visual inspection when stored overtime. However, some formulations of the invention advantageously do not exhibit noticeable settling over extended periods, for example 7 days or 14 days. As shown in Section 6, several exemplary formulations of Compound 1 did not exhibit noticeable settling when viewed at 7 and 14 days, whereas other formulations, particularly those including hydroxyethylcellulose (HEC) exhibited noticeable settling. Accordingly, in some embodiments, formulations of the invention do not include HEC.

In another aspect, the invention provides a kit comprising a formulation of Compound 1 described herein and a droptainer. A droptainer is a container comprising a bottle, tip and cap useful for dispensing a formulation as drops. Droptainers in various sizes are commercially available from various suppliers. Formulations of the invention can be in some embodiments packaged within droptainers, e.g., for single or multiple use. Various sizes of droptainers can be used, for example 4 ml or 8 ml droptainers, optionally having a 15 mm tip. Droptainer tips can be selected to provide a desired drop size, for example a tip that provides a drop size of 30 mg to 40 mg. In some embodiments, a kit of the invention comprises 4 ml of a formulation in a 8 ml droptainer. In other embodiments, a kit of the invention comprises 2 ml of a formulation in a 4 ml droptainer.

### 5.2.1. Compound 1 and Pharmaceutically Acceptable Salts Thereof

Compound 1 present in a formulation described herein can be in crystalline or amorphous form. Embodiments of the crystalline form of Compound 1 include the form designated as Form A. The names used herein to identify a specific form, e.g. “Form A”, etc., should not be considered limiting with respect to any other substance possessing similar or identical physical and chemical characteristics, but rather it should be understood that these designations are mere identifiers that should be interpreted according to the characterization information also presented herein. In certain embodiments, Form A is substantially pure.

In certain embodiments, formulations described herein comprise a Form A having an X-ray powder diffraction (XRPD) spectrum substantially the same as the XRPD shown in FIG. 1. In other embodiments, the formulations can comprise Form A characterized by an XRPD pattern comprising one or more peaks selected from 7.2±0.2, 7.8±0.2, 8.2±0.2, 10.7±0.2, 11.6±0.2, 12.5±0.2, 13.8±0.2, 14.5±0.2, 15.0±0.2, 15.8±0.2, 17.7±0.2, 18.9±0.2, 20.7±0.2, 21.3±0.2, 21.8±0.2, 22.1±0.2, and 23.1±0.2. Accordingly, the XRPD pattern for Form A may comprise one, two, three, or four representative peaks. In other embodiments, a formulation can comprise Form A characterized by an XRPD pattern comprising one or more peaks selected from FIG. 1, as shown in Table 1.

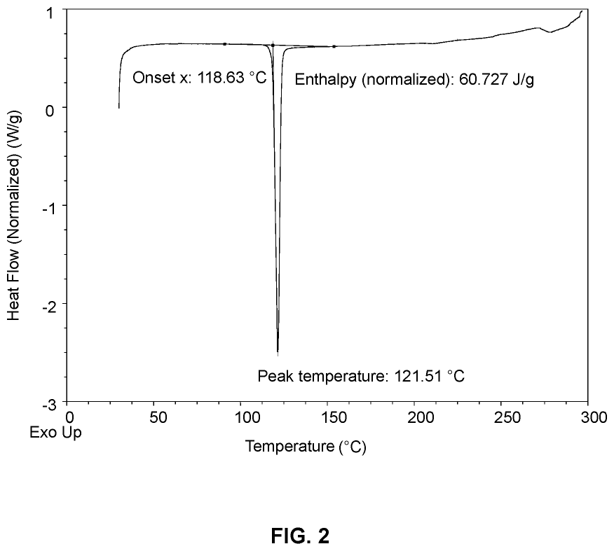

In certain embodiments, the formulations provided herein comprise Form A having a differential scanning calorimetry (DSC) profile substantially the same as the DSC profile shown in FIG. 2. In certain embodiments, the DSC profile is characterized by a single endothermic event representing the melting of the compound with a melting onset at about 118.6° C. at a heating rate of 10 K/min. In certain embodiments, the DSC profile is characterized by a single endothermic event at about 121.5° C. at a heating rate of 10 K/min which represents the melting of the compound.

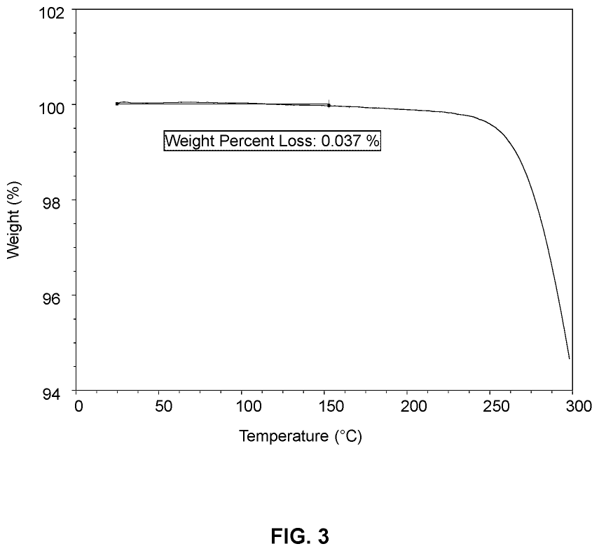

In certain embodiments, the formulations provided herein comprise Form A having a thermo gravimetric analysis (TGA) profile substantially the same as the TGA profile shown in FIG. 3. In certain embodiments, the weight loss represents a loss of about 0.04% of the sample as the temperature is changed from about 30° C. to about 150° C. In certain embodiments, the weight loss represents a loss of less than about 0.5% of the sample as the temperature is changed from about 30° C. to about 150° C.

In other embodiments, the formulations provided herein comprise an amorphous form of 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione.

It should be understood that in the XRPD spectra or pattern that there is inherent variability in the values measured in degrees 2theta (° 26) as a result of, for example, instrumental variation (including differences between instruments). As such, it should be understood that there is a variability of up to ±0.2° 2θ in XRPD peak measurements and yet such peak values would still be considered to be representative of a particular solid state form of the crystalline materials described herein. It should also be understood that other measured values from XRPD experiments and Karl Fisher analysis, such as relative intensity and water content, can vary as a result of, for example, sample preparation and/or storage and/or environmental conditions, and yet the measured values will still be considered to be representative of a particular solid state form of the crystalline materials described herein.

Formulations of the invention can in some embodiments include an amount of Compound 1 or a pharmaceutically acceptable salt thereof that has been sterilized. It was discovered that Compound 1 is relatively unstable when sterilized by steam and relatively stable when sterilized by gamma irradiation. Without being bound by theory, it is believed that the relatively instability observed for steam sterilization is due to the relatively low melting temperature of Compound 1. Thus, in some embodiments, the Compound 1 used in the formulations, processes, and methods of the invention is sterilized by radiation, for example gamma radiation or X-ray radiation. In some embodiments, Compound 1 used in the formulations, processes, and methods of the invention is sterilized by 10 kGy to 30 kGy of gamma radiation, for example 10 kGy to 20 kGy or 20 kGy to 30 kGy.

### 5.2.2. Pharmaceutically Acceptable Carriers

The formulations of the invention typically comprise one or more carriers selected from solubilizers, viscosity enhancers, tonicity enhancers, humectants, and pH adjusting agents. In some embodiments, the formulations of the invention comprise at least one solubilizer, at least one viscosity enhancer, at least one humectant, optionally at least tonicity enhancer, and optionally at least one pH adjusting agent. Exemplary solubilizers, viscosity enhancers, tonicity enhancers, humectants, and pH adjusting agents are described in Sections 5.2.2.1 to 5.2.2.5, respectively.

### 5.2.2.1. Solubilizers

Solubilizers are components of pharmaceutical formulations used to help solubilize one or more other components of the formulation, for example, an active pharmaceutical ingredient. Solubilizers include, but are not limited to, tyloxapol, fatty acid glycerol polyethylene glycol esters, fatty acid polyethylene glycol esters, polyethylene glycols, glycerol ethers, or cyclodextrins. As detailed in Section 6, infra, the solubility of Compound 1 was studied in various excipients, and it was discovered that the solubility of Compound 1 in castor oil-based solubilizers was much higher than in the other studied solubilizers. Accordingly, formulations of the invention typically include one or more castor oil-based solubilizers, for example polyoxyl 40 hydrogenated castor oil and/or polyoxyl 35 castor oil. In some embodiments, a formulation of the invention comprises a castor oil-based solubilizer which is polyoxyl 40 hydrogenated castor oil. In other embodiments, a formulation of the invention comprises polyoxyl 35 castor oil. The amount of castor oil-based solubilizers included in a formulation of the invention can range, for example, from 0.1% w/w to 5% w/w (e.g., at least 0.1% w/w, at least 0.2% w/w, at least 0.3% w/w, at least 0.4% w/w, or at least 0.5% w/w to no more than 5% w/w, no more than 4% w/w, no more than 3% w/w, no more than 2% w/w, or no more than 1% w/w). In some embodiments, the amount of castor-oil based solubilizer in a formulation (e.g., polyoxyl 40 hydrogenated castor oil/or polyoxyl 35 castor oil) is 0.5% or 1%.

### 5.2.2.2. Viscosity Enhancers

Formulations of the invention typically have a viscosity ranging from 50 cPs to 600 cPs. In some embodiments, the viscosity of a formulation is at least 50 cPs, at least 100 cPs, at least 150 cPs, at least 175 cPs, or at least 200 cPs and/or no more than 600 cPs, no more than 500 cPs, no more than 400 cPs, no more than 350 cPs, or no more than 300 cPs. In some embodiments, the viscosity of a formulation of the invention is 100 cPs to 400 cPs, 150 cPs to 400 cPs, or 200 cPs to 400 cPs.

Viscosity of a formulation can be adjusted by including one or more viscosity enhancing agents. Examples of viscosity enhancing agents include, but are not limited to, polysaccharides, such as hyaluronic acid and its salts, chondroitin sulfate and its salts, dextrans, various polymers of the cellulose family, vinyl polymers, and acrylic acid polymers. The formulations of the invention typically include a carbomer, which is an acrylic acid based polymer. In particular embodiments, the carbomer is carbomer homopolymer Type B, for example CARBOPOL® 974P (Carbomer 974P). The amount of homopolymer Type B included in a formulation of the invention can in some embodiments range from at least 0.1% w/w, at least 0.2% w/w, at least 0.3% w/w, at least 0.4% w/t at least 0.5% w/t to no more than 1% w/w, no more than 0.9% w/w, no more than 0.8% w/w, no more than 0.7% w/w, no more than 0.6% w/w or no more than 0.5% w/w. In some embodiments, the amount of carbomer homopolymer Type B is 0.5% w/w.

Salts such as sodium chloride tend to reduce the viscosity of carbomer-containing formulations. Thus, salts such as sodium chloride can be used to adjust the viscosity of a carbomer-containing formulation. In some embodiments, a formulation of the invention contains 0.1% to 0.5% w/w of sodium chloride (e.g., at least 0.1% w/w or at least 0.2% w/w to no more than 0.5% w/w, no more than 0.4% w/w, or no more than 0.3% w/w). In some embodiments, the amount of sodium chloride in a formulation of the invention is 0.25% w/w.

In some embodiments, a viscosity (or viscosity range) can be selected for a formulation of the invention so that the formulation has (1) sufficiently high viscosity so as to not run off of a fingertip and/or eyelid when administered thereto, and/or (2) sufficiently low viscosity so as to be easily dispensable from a droptainer or drop dispenser. In some embodiments, formulations of the disclosure form drops having a mass of 30 mg to 40 mg.

### 5.2.2.3. Tonicity Enhancers

The formulations of the invention typically have an osmolality ranging from 200 milliosmoles per kilogram (mOsm/kg) to 400 mOsm/kg (e.g., at least 200 mOsm/kg, at least 250 mOsm/kg, at least 275 mOsm/kg to no more than 400 mOsm/kg, nor more than 375 mOsm/kg, or no more than 350 mOsm/kg). In some embodiments, the osmolality of a formulation is 250 mOsm/kg to 350 mOsm/kg.

Tonicity of a formulation can be adjusted, if needed, by the use of tonicity enhancing agents. Such agents can be, for example, be of ionic and/or non-ionic type. Examples of ionic tonicity enhancers include, for example, alkali metal or earth metal halides such as, e.g., CaCl₂, KBr, KCl, LiCl, NaI, NaBr or NaCl, Na₂SO₄ or boric acid. Non-ionic tonicity enhancing agents include, e.g., urea, glycerin, sorbitol, mannitol, propylene glycol, or dextrose. In some embodiments, the formulations of the invention include an ionic tonicity enhancer, for example, NaCl.

### 5.2.2.4. Humectant

Formulations of the invention can in some embodiments include a humectant. Humectants can help a formulation of the invention retain water, which can increase the amount of time before an aqueous formulation dries when applied to skin (e.g., an eyelid). Without being bound by theory, it is believed that including a humectant in a formulation of the invention can improve skin permeation and/or enhance delivery of Compound 1 or a pharmaceutically acceptable salt thereof through skin. Exemplary humectants that can be used in the present invention include glycerin, propylene glycol, mannitol, and sorbitol. Humectants such as glycerin can also have emollient properties. The concentration of a particular humectant in a formulation can vary depending upon the agent selected. In some embodiments, formulations of the disclosure comprise glycerin, mannitol, or propylene glycol.

In some embodiments, formulations of the invention contain glycerin, for example in an amount ranging from 0.5% w/w to 3% w/w (e.g., at least 0.5% w/w or at least 1% w/w to no more than 3% w/w, or no more than 2% w/w). In some embodiments, the amount of glycerin in a formulation of the invention is 1.5% w/w.

In other embodiments, formulations of the invention contain mannitol, for example in an amount ranging from 1% w/w to 5% w/w (e.g., at least 1% w/w or at least 2% w/w to no more than 5% w/w or no more than 4% w/w). In some embodiments, the amount of mannitol in a formulation of the invention is 3% w/w.

### 5.2.2.5. pH Adjusting Agents

For the adjustment of the pH, e.g., to a physiological pH, acids and bases such as hydrochloric acid, sodium hydroxide, and tromethamine can be used. In certain embodiments, the pH of a formulation is adjusted to a desired pH with one or more of hydrochloric acid, sodium hydroxide and tromethamine. In some embodiments, the pH of a formulation of the invention is 6.5 to 7.5 (e.g. at least 6.5, at least 6.6, at least 6.7, at least 6.8, or at least 6.9 to no more than 7.5, no more than 7.4, no more than 7.3, no more than 7.2, or no more than 7.1). In some embodiments, the pH of a formulation of the invention is 6.9 to 7.1, e.g., 6.9, 7.0, or 7.1.

### 5.2.3. Exemplary Formulations and Kits

In various embodiments, formulations of the invention comprise Compound 1 or a pharmaceutically acceptable salt thereof, polyoxyl 40 hydrogenated castor oil, carbomer homopolymer type B, sodium chloride, and glycerin or mannitol, and water. If adjustment of pH is needed, a pH adjusting agent such as one or more of HCl, tromethamine, and sodium hydroxide can be used to adjust pH. In some embodiments, the formulations do not include a preservative. In some embodiments, the formulations includes a preservative.

In some embodiments, a formulation of the invention comprises:
a) 0.1% w/w to 3% w/w Compound 1 or a pharmaceutically acceptable salt thereof;
b) 0.1% w/w to 2.5% w/w polyoxyl 40 hydrogenated castor oil;
c) 0.1% w/w to 1% w/w carbomer homopolymer type B;
d) 0.1% w/w to 0.5% w/w sodium chloride;
e) 0.5% w/w to 3% w/w glycerin or 1% w/w to 5% w/w mannitol;
f) optionally, a pH adjusting agent such as HCl and/or tomethamine and/or NaOH in quantities sufficient (qs) to provide a pH of 6.8 to 7.2; and
g) water in a quantity sufficient (qs) to 100%.

In further embodiments, a formulation of the invention comprises:
a) 0.1% w/w to 3% w/w Compound 1 or a pharmaceutically acceptable salt thereof;
b) 0.5% w/w to 1% w/w polyoxyl 40 hydrogenated castor oil;
c) 0.5% w/w carbomer homopolymer type B;
d) 0.25% w/w sodium chloride;
e) 1.5% w/w glycerin or 3% mannitol;
f) optionally, a pH adjusting agent such as HCl and/or tomethamine and/or NaOH in quantities sufficient (qs) to provide a pH of 6.8 to 7.2; and
g) water in a quantity sufficient (qs) to 100%.

In further embodiments, a formulation of the invention comprises:
a) 0.1%, 0.3%, 1% or 3% w/w Compound 1;
b) 0.5% w/w polyoxyl 40 hydrogenated castor oil;
c) 0.5% w/w carbomer homopolymer type B;
d) 0.25% w/w sodium chloride;
e) 1.5% w/w glycerin;
f) optionally, a pH adjusting agent such as HCl and/or tomethamine and/or NaOH in quantities sufficient (qs) to provide a pH of 7.0; and
g) water in a quantity sufficient (qs) to 100%.

In further embodiments, a formulation of the invention comprises:
a) 0.1%, 0.3%, 1% or 3% w/w Compound 1;
b) 0.5% w/w polyoxyl 40 hydrogenated castor oil;
c) 0.5% w/w carbomer homopolymer type B;
d) 0.25% w/w sodium chloride;
e) 3% w/w mannitol;
f) optionally, a pH adjusting agent such as HCl and/or tomethamine and/or NaOH in quantities sufficient (qs) to provide a pH of 7.0; and
g) water in a quantity sufficient (qs) to 100%.

### 5.3. Processes for Making Formulations and Kits

In some aspects, the invention provides processes for making formulations and kits described herein. The processes of the invention typically comprise combining (a) a slurry comprising Compound 1 or a pharmaceutically acceptable salt thereof and one or more solubilizers such as a castor oil-based solubilizer with (b) one or more additional pharmaceutically acceptable carriers. For example, a formulation can be made by combining a slurry comprising Compound 1 and a solubilizer with a pre-made vehicle comprising all other components of the formulation.

The slurry can be produced, for example, by milling a mixture comprising the one or more solubilizers and Compound 1 or a pharmaceutically acceptable salt thereof to reduce the particle size of the Compound 1 or a pharmaceutically acceptable salt thereof to a desired size distribution. Milling can be performed until a desired D90 and/or D50 and/or D10 particle size is obtained, with longer milling times generally producing particles with smaller D90, D50, and D10 particle sizes. In some embodiments, the milling can be performed for at least 12, at least 18, or at least 24 hours. In some embodiments, milling is performed for up to 24 hours, or up to 36 hours.

In some embodiments, the milling is ball milling. Various sizes of beads can be used for ball milling, for example beads having a diameter of 1 mm to 5 mm. In some embodiments, the beads are 1 mm beads. In other embodiments, the beads are 3 mm. In some embodiments, the beads are zirconium beads.

In some embodiments, the slurry comprises Compound 1 or a pharmaceutically acceptable salt thereof that has been sterilized by irradiation, for example, gamma irradiation or X-ray radiation. In some embodiments, the slurry is made with Compound 1 or a pharmaceutically acceptable salt thereof that has been sterilized by gamma irradiation prior to formation of the slurry. Alternatively, the slurry comprising Compound 1 or a pharmaceutically acceptable salt thereof can be sterilized by gamma irradiation after the slurry is formed. Formation of the slurry and subsequent formulation steps can be performed under aseptic conditions. In some embodiments, a vehicle (e.g., comprising all components of a formulation other than the components used to make a slurry) can be steam sterilized prior to being combined with the slurry.

In some embodiments, a process of the invention for making a formulation comprises the step of combining the slurry with a vehicle comprising the remaining components of the formulation. In some embodiments, the process further comprises a step of making the slurry. Alternatively, preparation of the slurry can be separate from the process for making the complete formulation. For example, the slurry can be produced at one plant and combined with the vehicle at a different plant.

In some embodiments, the process can further comprise a step of making a kit described herein by filling a container, for example, a droptainer, with the formulation. Aseptic filling can be used.

### 5.4. Uses of Formulations and Kits

Without being bound by theory, it is believed that LXR agonists are useful in the treatment of ocular diseases and disorders such as dry eye disease and MGD. Accordingly, in one aspect, the invention provides a method of agonizing LXR in the meibomian glands of a subject (e.g., a subject having dry eye disease and/or MGD), comprising administering a therapeutically effective amount of a formulation of the invention to the subject.

In another aspect, the invention provides a method of inducing or increasing the expression of stearoyl-CoA desaturase-1 (SCD1) in the meibomian glands of a subject comprising administering a therapeutically effective amount of a formulation of the invention to the subject.

In another aspect, the invention provides a method of increasing the ratio of desaturated lipids to saturated lipids in the eye a subject comprising administering a therapeutically effective amount of a formulation of the invention to the subject.

In another aspect, the invention provides a method of lowering a subject's meibum melting temperature and/or increasing meibum outflow from a subject's meibomian glands comprising administering a therapeutically effective amount of a formulation of the invention to the subject.

In another aspect, the invention provides a method of reducing obstruction of meibum outflow from a subject's meibomian glands comprising administering a therapeutically effective amount of a formulation of the invention to the subject.

In some embodiments of the methods described above in this Section, the subject has dry eye disease, for example evaporative dry eye disease, and/or Meibomian gland dysfunction.

In certain aspects, provided herein is a method for treating an ocular disease or disorder, wherein the method comprises administering a therapeutically effective amount of a formulation of the invention to a subject in need thereof.

In certain aspects, provided herein is a method for treating the signs and/or symptoms of an ocular disease or disorder, wherein the method comprises administering a therapeutically effective amount of a formulation of the invention to a subject in need thereof.

In certain embodiments, provided herein is a method for treating evaporative dry eye disease, which can be caused by Meibomian gland dysfunction, wherein the method comprises administering a formulation of the invention to a subject in need thereof.

In certain embodiments, provided herein is a method for treating Meibomian gland dysfunction, wherein the method comprises administering a formulation of the invention to a subject in need thereof.

In certain embodiments, provided herein is a method for treating the signs and/or symptoms of Meibomian gland dysfunction, wherein the method comprises administering a therapeutically effective amount of a formulation of the invention to a subject in need thereof.

Formulations of the invention can in some embodiments be administered to a subject's eyelids. Application of the pharmaceutical composition may be performed with an applicator, such as the subject's finger, a Weck-Cel®, Q-tip®, or other device capable of delivering a formulation to the eyelid in order to deliver the formulation to the meibomian gland.

In some embodiments, the symptoms of a patient are assessed by asking the patient a series of questions. Questionnaires allow the assessment of a range of symptoms associated with ocular discomfort. In some embodiments, the questionnaire is the SPEED questionnaire. The SPEED questionnaire assesses frequency and severity of a patient's dry eye symptoms. It examines the occurrence of symptoms on the current day, past 72 hours and past three months. A SPEED score is tallied based on the patient's answers to the questions, to give a range of severity of the patient's symptoms. The SPEED questionnaire includes questions such as the following: 1) what dry eye symptoms are you experiencing, and when do they occur? 2) how frequently do you experience dryness, grittiness, or scratchiness in your eyes? 3) how often do you experience soreness or irritation of the eyes? 4) how often do you experience burning or watering of the eyes? 5) how often do you experience eye fatigue? and 6) how severe are the symptoms? In some embodiments, the questionnaire is the IDEEL questionnaire, which is similar to the SPEED questionnaire described above.

Meibomian gland expressibility is optionally determined to assess the meibomian gland function. In normal patients, meibum is a clear to light yellow oil. Meibum is excreted from the glands when digital pressure is placed on the glands. Changes in meibomian gland expressibility are one potential indicator of MGD. In some embodiments, during expression, quantifying the amount of physical force applied during expression is monitored in addition to assessing lipid volume and lipid quantity.

Tear stability break up time (TBUT) is a surrogate marker for tear stability. Tear film instability is a core mechanism in dry eye and MGD. Low TBUT implies a possibility of lipid layer compromise and MGD. TBUT is optionally measured by examining fluorescein breakup time, as defined as the time to initial breakup of the tear film after a blink. Fluorescein is optionally applied by wetting a commercially available fluorescein-impregnated strip with saline, and applied to the inferior fornix or bulbar conjuctiva. The patient is then asked to blink several times and move the eyes. The break up is then analyzed with a slit lamp, a cobalt blue filter, and a beam width of 4 mm. The patient is instructed to blink, and the time from upstroke of the last blink to the first tear film break or dry spot formation is recorded as a measurement.

Other methods for assessing MGD signs and/or symptoms, include but are not limited to, Schirmer test, ocular surface staining, lid morphology analysis, meibography, meibometry, interferometry, evaporimetry, tear lipid composition analysis, fluorophotometry, meiscometry, lipid layer thickness, meibum desaturation index, meibomian gland loss osmolarity analysis, indices of tear film dynamics, reading speed, evaporation and tear turnover. Analysis of MGD signs and/or symptoms is performed by commonly understood methods known to those of skill in the art.

In some embodiments of the present invention, the subject is diagnosed with Meibomian gland dysfunction or dry eye disease or ocular surface disease.

In some embodiments, the administration decreases the signs and/or symptoms of Meibomian gland dysfunction or dry eye disease or ocular surface disease. In particular embodiments, the administration of a formulation of the invention results in one or more of the following (or similar or equivalent tests):
i) increased tear film break-up time of at least 2, 3, 4, or 5 seconds;
ii) meibomian gland expression grading improvement by 1 or 2 or 3 grades;
iii) increased tear meniscus of at least 10%;
iv) decreased corneal fluorescein staining of at least 10%, or
v) increased Schirmer test score of at least 2 mm.

As used herein, “meibomian gland expression grading” refers to a scale for assessing the severity of Meibomian gland dysfunction, for example, as described in Tomlinson, Alan, et al. (2011), “The International Workshop on Meibomian Gland Dysfunction: Report of the Diagnosis Subcommittee,” Investigative Ophthalmology & Visual Science, vol. 52, no. 4, pp. 2006-2049.

In additional aspects, the invention provides formulations provided herein for use in a method described herein, for example, in the methods described in this Section 5.4.

### 6. EXAMPLES

The following examples are intended to illustrate the invention and are not to be construed as being limitations thereon. Temperatures are given in degrees centigrade. The structure of final products, intermediates and starting materials is confirmed by standard analytical methods, e.g., microanalysis and spectroscopic characteristics, e.g., MS, IR, and NMR. Abbreviations used are those conventional in the art. If not defined, the terms have their generally accepted meanings.

6.1. Abbreviations

| | |
|---|---|
| aq.: aqueous | M and mM: molar and millimolar |
| br: broad | mg: milligram |
| d: doublet; dd: doublet of doublets; | m/z: mass to charge ratio |
| ddd: doublets of doublet of doublets | |
| DCM: dichloromethane | NMR: nuclear magnetic resonance |
| DMF: dimethylformamide | q: quartet |
| EtOAc: ethyl acetate | s: singlet |
| HPLC: high pressure liquid | t: triplet |
| chromatography | td: triplet of doublets |
| | tt: triplet of triplets |
| h, hr: hour(s) | wt: weight |
| LC and LCMS: liquid chromatog- | μL, mL and L: microliter, |
| raphy and liquid chromatography- | milliliter and liter |
| mass spectrometry | |
| m: multiplet | Rt.: retention time |
| min(s): minute(s) | mCPBA: m-chloroperoxybenzoic |
| | acid |

### 6.2. Analytical Methods

### LCMS Method 1:

Instrument: Waters AcQuity UPLC; column: AcQuity UPLC BEH C₁₈ 1.7 μm, 2.1×30 mm; 2 min run time, 2% solvent B from 0 to 0.1 min, 2→98% solvent B: solvent A from 0.1 to 1.8 min, 98% solvent B for 0.2 min. Solvents: solvent A=0.1% formic acid in water (v/v), solvent B=0.1% formic acid in acetonitrile (v/v). UV detection array 210-400; mass detection 120-1250; column at 50° C.; flow rate 1.0 mL/min; pH 2.6.

### LCMS Method 2:

Instrument: Waters AcQuity UPLC; column: AcQuity UPLC BEH C₁₈ 1.7 μm 2.1×30 mm; 2 min run time, 2% solvent B from 0 to 0.1 min, 2→98% solvent B: solvent A from 0.1 to 1.8 min, 98% solvent B for 0.2 min. Solvents: solvent A=5 mM ammonium hydroxide in water, solvent B=5 mM ammonium hydroxide in acetonitrile. UV detection array 210-400; mass detection 120-1250; column at 50° C.; flow rate 1.0 mL/min; pH 10.2.

### LCMS Method 3:

Instrument: Waters AcQuity UPLC; column AcQuity UPLC BEH C18 1.7 μm 2.1×50 mm; 5.2 min run time, 2→98% solvent B: solvent A from 0 to 5.15 min, 98% solvent B from 5.15 to 5.20 min. Solvents: solvent A=5 mM ammonium hydroxide in water, solvent B=5 mM ammonium hydroxide in acetonitrile. UV detection array 210-400; mass detection 120-1600; column at 50° C.; flow rate 1.0 mL/min; pH 10.2.

### 6.3. Example 1: Synthesis of 3-((3-(4-(2-(isobutylsulfonyl)phenoxy)-3- (trifluoromethyl)phenyl)-1,2,4-oxadiazol-5-yl)methyl)-5,5-dimethyl-1-(2-morpholinoethyl)imidazolidine-2,4-dione (Compound 1)

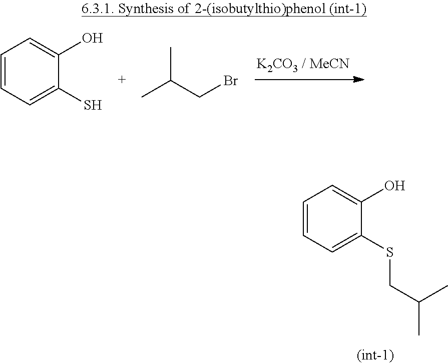

2-mercaptophenol (8.00 mL, 79 mmol) was added to a stirred suspension of potassium carbonate (21.91 g, 159 mmol) in acetonitrile (200 mL) at room temperature. After 10 min, 1-bromo-2-methylpropane (9.48 mL, 87 mmol) was added. The mixture was then stirred at room temperature overnight. Additional portions of 0.3 eq of 1-bromo-2-methylpropane and 0.5 eq of potassium carbonate were added and the mixture was further stirred for one more day. After concentration in vacuo, the residue was dissolved in DCM and filtered through Celite® to remove solid potassium carbonate. The filtrate was purified by column chromatography (0-20% EtOAc/heptane) to provide 2-(isobutylthio)phenol (int-1). ¹H NMR (400 MHz, CD2Cl₂) 6 ppm 7.50 (dd, J=7.70, 1.59 Hz, 1H) 7.25-7.32 (m, 1H) 6.99 (dd, J=8.13, 1.16 Hz, 1H) 6.90 (td, J=7.52, 1.34 Hz, 1H) 6.76 (s, 1H) 2.64 (d, J=6.97 Hz, 2H) 1.72-1.88 (m, 1H) 1.04 (d, J=6.60 Hz, 6H).

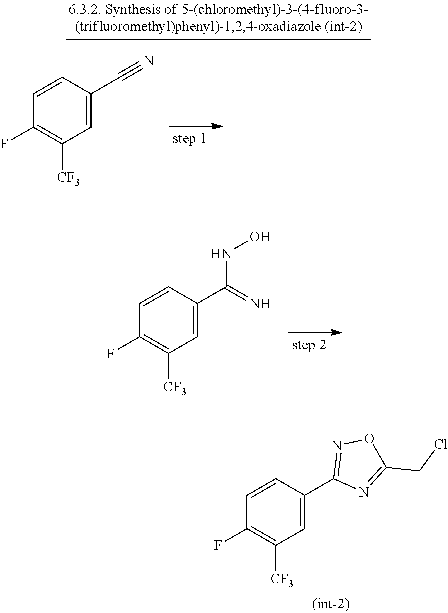 [SMILES: N#Cc1ccc(F)c(C(F)(F)F)c1 ; N=C(NO)c1ccc(F)c(C(F)(F)F)c1 ; Fc1ccc(-c2noc(CCl)n2)cc1C(F)(F)F]

Step 1: In a 500 mL 3 neck flask, to a solution of 4-fluoro-3-(trifluoromethyl)benzonitrile (15.0 g, 79.32 mmol) in methanol (150 mL) was added a hydroxylamine solution 50 wt. % in water (52.4 mL, 793.2 mmol) dropwise at room temperature. The reaction mixture was stirred at room temperature for 16 hours, then concentrated in vacuo and the residue diluted with water (100 mL). The mixture was extracted with ethyl acetate (3×150 mL) and the combined organic layers dried over sodium sulfate, filtered, and concentrated to provide 4-fluoro-N-hydroxy-3-(trifluoromethyl)benzimidamide. LCMS Method 2: Rt.=0.97 min.; m/z 223.1 [M+H]+; 1H NMR (400 MHz, (CD₃)₂SO) δ 9.88 (s, 1H), 8.02-8.03 (m, 2H), 7.51-7.56 (m, 1H), 6.06 (s, 2H).

Step 2: In a 500 mL 3 neck flask, to a solution of 4-fluoro-N-hydroxy-3-(trifluoromethyl)benzimidamide (16.0 g, 72.0 mmol) in toluene (160 mL) was added chloroacetyl chloride (17.2 mL, 216.2 mmol) dropwise at 0° C. After completion of addition, the turbid yellow reaction mixture was heated to reflux for 6 hours. The clear reaction solution was then allowed to cool to room temperature and quenched with ice-cold water. The organic layer was washed with saturated sodium bicarbonate solution (100 mL) and brine (100 mL); the dried over sodium sulfate, filtered, and concentrated. The crude product was purified by column chromatography using 3-6% ethyl acetate-hexane to obtain 5-(chloromethyl)-3-(4-fluoro-3-(trifluoromethyl)phenyl)-1,2,4-oxadiazole (int-2). 1H NMR (400 MHz, (CD₃)₂SO) δ 8.38-8.39 (m, 1H), 8.25-8.27 (m, 1H), 7.73-7.78 (m, 1H), 5.24 (s, 2H).

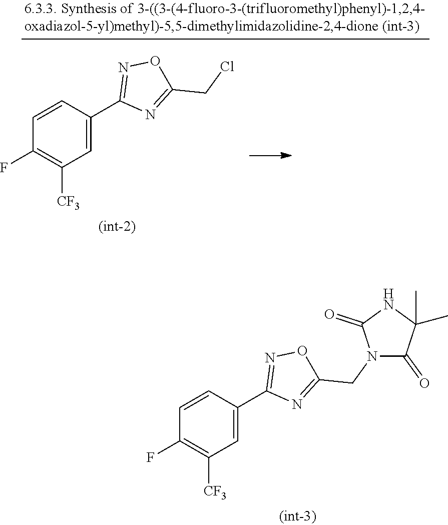 [SMILES: Fc1ccc(-c2noc(CCl)n2)cc1C(F)(F)F ; CC1(C)NC(=O)N(Cc2nc(-c3ccc(F)c(C(F)(F)F)c3)no2)C1=O]

A solution of 5-(chloromethyl)-3-(4-fluoro-3-(trifluoromethyl)phenyl)-1,2,4-oxadiazole (int-2) (4.35 g, 15.50 mmol) in DMF (25 mL) was treated with 5,5-dimethylimidazolidine-2,4-dione (2.98 g, 23.25 mmol) and potassium carbonate (4.28 g, 31.0 mmol). The reaction mixture was stirred at 60° C. for 2 hours and then poured into ice water (100 mL) and refrigerated overnight. The precipitate was collected and washed with ice-water to give 3-((3-(4-fluoro-3-(trifluoromethyl)phenyl)-1,2,4-oxadiazol-5-yl)methyl)-5,5-dimethylimidazolidine-2,4-dione (int-3). LCMS Method 1: m/z 373.2 [M+H]+; 1H NMR (400 MHz, CDCl₃) δ 8.29 (dd, J=6.7, 1.9 Hz, 1H), 8.23 (ddd, J=8.4, 4.6, 2.1 Hz, 1H), 7.35-7.27 (m, 1H), 5.00 (s, 2H), 1.52 (s, 6H).

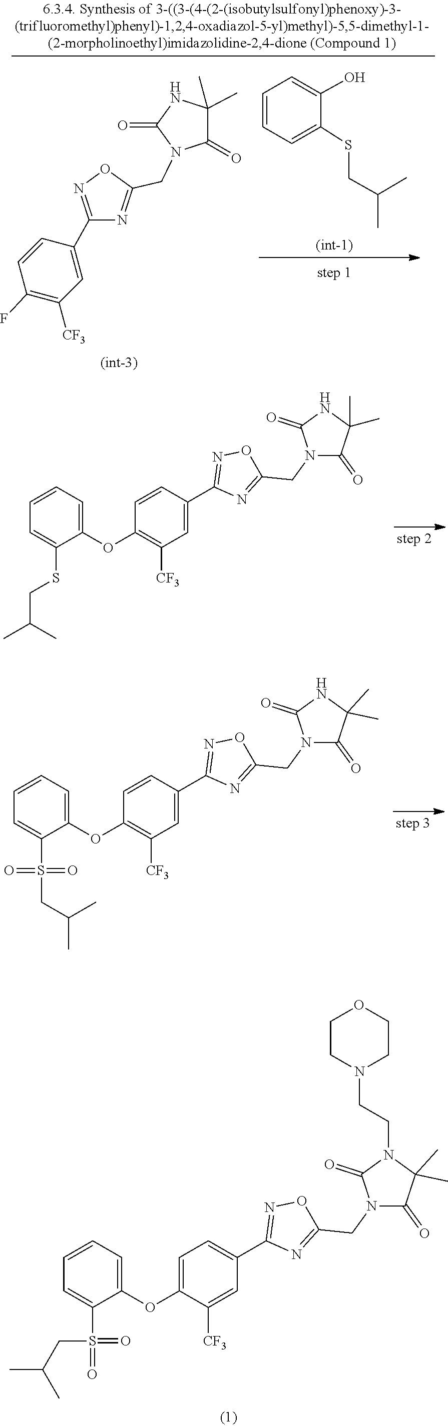

Step 1: To a solution of 3-((3-(4-fluoro-3-(trifluoromethyl)phenyl)-1,2,4-oxadiazol-5-yl)methyl)-5,5-dimethylimidazolidine-2,4-dione (int-3) (7.26 g, 39.8 mmol) in DMF (100 mL) was added 2-(isobutylthio)phenol (int-1) (4.94 g, 13.27 mmol) and potassium carbonate (5.50 g, 39.8 mmol). The mixture was heated at 90° C. overnight, then left to cool to room temperature, and then diluted with saturated ammonium chloride solution. The mixture was extracted with EtOAc, and the organic layer washed with brine, dried over sodium sulfate, filtered, and concentrated. The residue was purified by flash column chromatography (0-60% EtOAc/Heptanes) to provide: 3-((3-(4-(2-(isobutylthio)phenoxy)-3-(trifluoromethyl)phenyl)-1,2,4-oxadiazol-5-yl)methyl)-5,5-dimethylimidazolidine-2,4-dione. LCMS Method 3: m/z [M+]=535.4. 1 H NMR (400 MHz, CD₂Cl₂) δ 8.39 (d, J=2.0 Hz, 1H), 8.11 (dd, J=8.7, 2.1 Hz, 1H), 7.49 (dd, J=5.8, 3.5 Hz, 1H), 7.34-7.19 (m, 2H), 7.13-6.95 (m, 1H), 6.80 (d, J=8.8 Hz, 1H), 5.77 (s, 1H), 4.99 (s, 2H), 2.82 (d, J=6.9 Hz, 2H), 1.84 (dh, J=13.3, 6.8 Hz, 1H), 1.55 (s, 6H), 1.01 (d, J=6.8 Hz, 6H).

Step 2: mCPBA (8.43 g, 37.6 mmol) was added to a mixture of 3-((3-(4-(2-(isobutylthio)phenoxy)-3-(trifluoromethyl)phenyl)-1,2,4-oxadiazol-5-yl)methyl)-5,5-dimethylimidazolidine-2,4-dione (6.7 g, 12.53 mmol) in dichloromethane (200 mL). After stirring for 1 hour, saturated sodium bicarbonate solution was added. The organic layer was washed with brine, dried over sodium sulfate, filt

[symbol]

entrated. The residue was purified by flash column chromatography (0-100% EtOAc/heptanes) to provide 3-((3-(4-(2-(isobutylsulfonyl)phenoxy)-3-(trifluoromethyl)phenyl)-1,2,4-oxadiazol-5-yl)methyl)-5,5-dimethylimidazolidine-2,4-dione. LCMS Method 3: m/z [M+H]+ 567.2; 1H NMR (400 MHz, (CD₃)₂SO) δ 8.55 (s, 1H), 8.28 (d, J=2.1 Hz, 1H), 8.25 (dd, J=8.7, 2.2 Hz, 1H), 8.03 (dd, J=7.9, 1.7 Hz, 1H), 7.82 (ddd, J=8.3, 7.5, 1.7 Hz, 1H), 7.57 (td, J=7.6, 1.0 Hz, 1H), 7.33 (dd, J=8.2, 1.0 Hz, 1H), 7.15 (d, J=8.7 Hz, 1H), 5.02 (s, 2H), 3.36 (d, J=6.5 Hz, 2H), 2.07 (hept, J=6.6 Hz, 1H), 1.38 (s, 6H), 0.98 (d, J=6.7 Hz, 6H).

Step 3: 4-(2-bromoethyl)morpholine (7.09 g, 25.8 mmol) was added to a mixture of 3-((3-(4-(2-(isobutylsulfonyl)phenoxy)-3-(trifluoromethyl)phenyl)-1,2,4-oxadiazol-5-yl)methyl)-5,5-dimethylimidazolidine-2,4-dione (7.3 g, 12.88 mmol) and cesium carbonate (14.69 g, 45.1 mmol) in DMF (100 mL). The mixture was stirred overnight. The mixture was then diluted with ethyl acetate, then washed sequentially with water and brine, dried over sodium sulfate, filtered and concentrated. The residue was purified by flash column chromatography (0-100% EtOAc/Heptane, then 1% triethylamine in EtOAc) to provide 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione (1). LCMS Method 3: Rt.=2.81 mins.; m/z [M+H]+ 680.5. ¹H NMR (400 MHz, CDCl3) δ 8.38 (d, J=2.1 Hz, 1H), 8.20-8.10 (m, 2H), 7.64 (ddd, J=8.2, 7.4, 1.7 Hz, 1H), 7.41 (td, J=7.6, 1.1 Hz, 1H), 7.04-6.97 (m, 2H), 4.99 (s, 2H), 3.71 (br s, 4H), 3.48 (s, 2H), 3.35 (d, J=6.6 Hz, 2H), 2.65 (s, 2H), 2.56 (s, 4H), 2.26 (hept, J=6.7 Hz, 1H), 1.52 (s, 6H), 1.07 (d, J=6.7 Hz, 6H).

### 6.4. Example 2: Characterization of Compound 1 Form A

### 6.4.1. Preparation of 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione

A 100 mg/mL solution of 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione was prepared in ethanol solution at 50° C. The clear solution was then slowly cooled to room temperature. Upon completion of precipitate formation, the resulting solids were collected using a filter and dried to provide Form A. The characterization of Form A was conducted using X-ray powder diffraction (XRPD), differential scanning calorimetry (DCA), and thermogravimetry analysis (TGA) techniques.

### 6.4.2. X-Ray Powder Diffraction

The X-ray powder diffraction (XRPD) data was collected on a D8 Advance diffractometer using CuKα1 radiation (1.54056 Å) with germanium monochromator at room temperature. The data were collected from 3 to 45° 2θ. Detector scan on solid state LynxEye detector was performed using 0.020 per step with 19.2 s/step scan speed. The sample was measured on a zero background silicon wafer. FIG. 1 illustrates the XRPD of Form A.

TABLE 1
X-ray powder diffraction data for Form A
Angle/°2theta

7.2
7.8
8.2
10.7
11.6
12.5
13.8
14.5
15.0
15.8
17.7
18.9
20.7
21.3
21.8
22.1
23.1

### 6.4.3. Differential Scanning Calorimetry

Melting properties of Form A were obtained from differential scanning calorimetry (DCA) thermograms, recorded with a TA Discovery Q5000 (ThermoAnalytical). Samples were sealed in standard 40 μl aluminum pans, pin-holed and heated in the DSC from 30° C. to 300° C., at a heating rate of 10 K/min. Dry N₂ gas, at a flow rate of 50 mL/min was used to purge the DSC equipment during the measurement. FIG. 2 illustrates the DSC profile of Form A.

The DSC curve of Form A (FIG. 2) shows a single endothermic peak with an onset temperature of about 118.6° C. and a peak temperature of about 121.5° C., which is due to the melting of the sample.

### 6.4.4. Thermogravimetric Analysis

Form A was analyzed using thermogravimetry analysis (TGA). Loss on drying was determined by TGA using a TA Discovery Q2000 (ThermoAnalytical), resulting in a weight vs. temperature curve. Samples were weighed into 100 μL aluminum crucibles and sealed. The seals were pin-holed and the crucibles heated in the TGA from 30° C. to 300° C. at a heating rate of 10 K/min. Dry N₂ gas was used for purging. FIG. 3 illustrates the TGA profile of Form A.

The TGA curve (FIG. 3) shows no significant mass loss until the sample melts. For example mass losses of only about 0.04 weight % up to a temperature of about 150° C. was observed.

### 6.5. Example 3: Biological Activity of Compound 1

The LXR agonist Compound 1 provided herein was shown to induce stearoyl-CoA desaturase-1 (SCD1) expression thereby increasing lipid desaturation of meibum and reducing meibum melting temperature which can lead to reduced tear film evaporation and ameliorating the symptoms of Meibomian gland dysfunction and evaporative dry eye disease. To investigate the effect of Compound 1, SCD1 expression following treatment with Compound 1 was evaluated. Changes in the global desaturation index in SZ95 sebocytes upon administration of Compound 1 was also investigated. Finally, in vivo measurement of meibum melting temperature was investigated for Compound 1.

### 6.5.1. Measurement of SCD1 Expression in SZ95-SCD1-HiBit Cells

SCD1 expression levels in SZ95 cells were quantitated using the HiBiT system (Promega) in which a small peptide sequence (SmBiT) with a larger protein (LgBiT) reconstitutes luciferase activity and can generate a luminescent signal used for quantitation. SZ95-SCD1-HiBit cells were generated by editing the SCD1 gene of SZ95 cells resulting in the addition of an 8 amino acid linker sequence (gssggssg, SEQ ID NO:1) followed by an 11-amino acid SmBiT sequence (vsgwrlfkkis, SEQ ID NO:2) at the carboxy-terminus of the SCD1 protein. The gRNA targeting sequence used was actacaagagtggctgagtt (SEQ ID NO: 3) and the SCD1 insertion oligonucleotide encoding the SmBiT sequence was:

1:
(SEQ ID NO: 4)
5′gaagaaagtctccaaggccgccatcttggccaggattaaaagaaccg
gagatggaaactacaagagtggcggtagtagtggtggtagtagtggtgt
gagcggctggcggctgttcaagaagattagctgagtttggggtccctca
ggttcctttttcaaaaaccagccaggcagaggttttaatgtctgtttat
taacta-3′.

The DNA sequence encoding the SCD1 with the linker and SmBiT tag at the carboxy-terminal tail of the protein is:

(SEQ ID NO: 5)
Atgccggcccacttgctgcaggacgatatctctagctcctataccacca
ccaccaccattacagcgcctccctccagggtcctgcagaatggaggaga
taagttggagacgatgcccctctacttggaagacgacattcgccctgat
ataaaagatgatatatatgaccccacctacaaggataaggaaggcccaa
gccccaaggttgaatatgtctggagaaacatcatccttatgtctctgct
acacttgggagccctgtatgggatcactttgattcctacctgcaagttc
tacacctggctttggggggtattctactattttgtcagtgccctgggca
taacagcaggagctcatcgtctgtggagccaccgctcttacaaagctcg
gctgcccctacggctctttctgatcattgccaacacaatggcattccag
aatgatgtctatgaatgggctcgtgaccaccgtgccc [symbol]
gaaacacatgctgatcctcataattcccgacgtggctttttcttctctc
acgtgggttggctgcttgtgcgcaaacacccagctgtcaaagagaaggg
gagtacgctagacttgtctgacctagaagctgagaaactggtgatgttc
cagaggaggtactacaaacctggcttgctgatgatgtgcttcatcctgc
ccacgcttgtgccctggtatttctggggtgaaacttttcaaaacagtgt
gttcgttgccactttcttgcgatatgctgtggtgcttaatgccacctgg
ctggtgaacagtgctgcccacctcttcggatatcgtccttatgacaaga
acattagcccccgggagaatatcctggtttcacttggagctgtgggtga
gggcttccacaactaccaccactcctttccctatgactactctgccagt
gagtaccgctggcacatcaacttcaccacattcttcattgattgcatgg
ccgccctcggtctggcctatgaccggaagaaagtctccaaggccgccat
cttggccaggattaaaagaaccggagatggaaactacaagagtggcggt
agtagtggtggtagtagtggtgtgagcggctggcggctgttcaagaaga
ttagctga. [symbol]
indicates data missing or illegible when filed

SZ95-SCD1-HiBit cells were seeded in 384-well cell culture white plates at a density of 3000 cells/30 d. Water was added to edge wells to avoid evaporation. Cells were incubated in a humidified incubator with 5% CO₂ at 37° C. overnight. Tested compounds were diluted at a ratio of 1:3 in DMSO using an Agilent BRAVO Automated Liquid Handling Platform and, after further serial dilutions, added to cells at final concentrations starting from 18 μM. 2-(3-(3-((2-chloro-3-(trifluoromethyl)benzyl)(2,2-diphenylethyl)amino)propoxy)phenyl)acetic acid was used as a reference compound in each plate. Cells in the assay plate were incubated in a humidified incubator with 5% CO₂ at 37° C. for 48h.

The assay plates were removed from the incubator and allowed to equilibrate to room temperature. Nano-Glo® HiBiT Detection Reagent (Promega; a mixture of Nano-Glo HiBiT Detection Buffer, Nano-Glo HiBiT Detection Substrate, and LgBiT protein, according to the manufacturer's instructions) was added into assay plates, at a volume equal to cell culture medium in each well. Plates were placed on an orbital shaker at a speed of 300-600 rpm for 10 min at room temperature, and read on an EnVision Plate Reader using luminescence detection with a 1 second read time.

The assay measures the increase in SCD1 protein production in vitro. Results are shown in Table 2 below. Aₘₐₓ refers to the percent EC₅₀ of the tested compound compared to a reference compound, 2-(3-(3-((2-chloro-3-(trifluoromethyl)benzyl)(2,2-diphenylethyl)amino)propoxy)phenyl)acetic acid.

### 6.5.2. Sentinel Lipid Assay (SLA)

The sentinel lipid assay was used quantify the change in the global desaturation index in SZ95 sebocytes upon administration of Compound 1. The assay measures a smaller subset of lipid analytes in meibum (termed “sentinel lipids”) which would model the global changes the population of both saturated and desaturated lipids in the cells. In order to define this smaller subset of lipids, a complete lipid profile was recorded on dose response curves (eight levels from 4 nM to 10 uM) of Compound 1. An elastic net regression model was applied separately to both the saturated and desaturated lipids to determine the minimum combination of coefficients and analytes, which could be used to adequately model the total population of lipids. The elastic net model was able to reduce the behavior of 425 lipids to 11 lipids and the correlation between the desaturation indices observed using the complete set of lipids with from the 11 sentinel lipids was 0.96.

A medium throughput assay was created using this reduced set of sentinel lipids. A single batch is defined as triplicate examples of three unique plates (i.e., a single batch of cells is used to create nine plates for LC-MSMS analysis). Lipids were extracted from the cells using a 1:1 mixture of methylene chloride/methanol containing 10 nM of deuterated standards of triglycerides, which are used as internal standards for quantitating the lipid abundance. The lipids were separated prior to mass spectrometric analysis using a five minute HPLC gradient. The abundance of the sentinel lipids and the internal standards are measured using multiple reaction monitor mode (MRM) on a triple quadrupole mass spectrometer. The data was transformed from total ion current to nmoles/10⁶ cells, which are multiplied by the coefficients from the elastic net model to determine the effective desaturated and saturated content, and therein the desaturation index of the dosed cells. In order to compare compounds from multiple batches with one another, the measure raw desaturation index was normalized by dividing it by the desaturation index of the DMSO dosed cells, and all data was assessed as the fraction by which the compound increases the desaturation index above 1.

SZ95 (immortalized human sebaceous gland cells) cells were seeded in Greiner bio-one 96-well polypropylene plates that were pre-treated with 50 μg/mL Human Plasma Fibronectin (Thermo Fisher Scientific) at a density of 10⁴ cells/135 μl. Cells were incubated in a humidified incubator with 5% CO₂ at 37° C. overnight. Test compounds were diluted at a ratio of 1:3 and added to cells at final concentrations starting from 10 μM. 2-(3-(3-((2-chloro-3-(trifluoromethyl)benzyl)(2,2-diphenylethyl)amino)propoxy)phenyl)acetic acid was used as a positive control reference compound in each plate. Cells in the assay plate were incubated in a humidified incubator with 5% CO₂ at 37° C. for 72h.

Culture medium was removed from the cells and cells in culture plates were washed with ice cold phosphate buffered saline three times. Plates were heat sealed and stored in a −80° C. freezer prior to Sentinel lipid assay. Results from the sentinel lipid assay are shown in Table 2. The Aₘₐₓ value refers to the percent EC₅₀ of the tested compound compared to a reference compound, 2-(3-(3-((2-chloro-3-(trifluoromethyl)benzyl)(2,2-diphenylethyl)amino)propoxy)phenyl)acetic acid.

TABLE 2
Results from HiBiT assay and Sentinal Lipid Assay (SLA)

| | HiBit | | | SLA | | |
|---|---|---|---|---|---|---|
| Com- | EC₅₀ | | Normalized | EC₅₀ | | Normalized |
| pound | (nM) | Amax | Amax^{b} | (nM) | Amax | Amax^{b} |
| 1 | 0.217 | 84.1 | 88.4 | 4.6 | 168.6 | 175.6 |
| Reference | 65.400 | 95.1 | 100.0 | 87.3 | 96.0 | 100.0 |

n.d. indicates not determined and the lowest dose used in the SLA assay was 4.6 nM.
^{a}indicates that the initial dilution for the compound was 1:100 in assay medium rather than 1:3 in DMSO.
The lowest dose used in the SLA assay was 4.6 nM.
^{b}indicates normalized Amax values based on the reference compound Amax of 100%.

This data indicates that Compound 1 robustly upregulates SCD1 protein production in SZ95 cells and in turn increase the desaturation index of the lipids these cells produce. By increasing the desaturation index, the viscosity of meibum may be lowered leading to better meibum outflow in vivo and ameliorating the signs and symptoms of Meibomian gland dysfunction and evaporative dry eye disease. These data indicate the potential for Compound 1 to lower meibum melting temperature in vivo and thereby ameliorate symptoms of evaporative dry eye disease (e.g., dry eye associated with MGD) and MGD.

### 6.5.3. In Vivo Measurement of Lowered Meibum Melting Temperature

The lowering of rat meibum melting temperature by Compound 1 was measured in naive Sprague Dawley rats. The test animals were administered either vehicle, or a suspension of 1% of Compound 1, as eye drops twice a day for fourteen days. The rat meibum was collected after administration of the compounds and analyzed by differential scanning calorimetry to measure the melting point. The lowering of meibum melting point in rats administered Compound 1 was compared to the vehicle.

Melting properties of meibum were obtained using differential scanning calorimetry thermograms, recorded on a TA Discovery Q5000 (ThermoAnalytical). Samples were sealed in standard 40 μl aluminum pans and subjected to a heat-cool-heat cycle with melting temperatures being recorded on the second heating ramp. The samples were first heated to 150° C. at 30K/min then cooled −30° C. at 30K/min. Next, the sample was heated to 75° C. with an underlying heating rate of 2K/min, a period of modulation of 60 seconds and a temperature amplitude of modulation of 1° C. Dry N₂ gas, at a flow rate of 50 mL/min was used to purge the DSC equipment during the measurement. Onset of melting and peak temperatures were recorded with peak temperature quoted as the melting point.

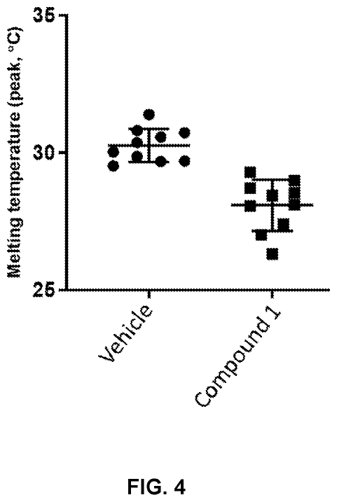

Results from the assay are shown Table 3 and in FIG. 4. Results were analyzed using an unpaired t test with Welch's correction.

TABLE 3
Results from in vivo administration of Compound 1

| | |
|---|---|
| P value | <0.0001 |
| Mean ± SEM of Vehicle | 30.27 ± 0.1917 |
| Mean ± SEM of Compound | 28.08 ± 0.2945 |
| Change of Tm | −2.181 ± 0.3731 |

As seen in the results presented herein, Compound 1 lowered meibum melting temperature in vivo and increased meibum desaturation index in vitro.

### 6.6. Example 4: Compound 1 is Chemically Stable when Sterilized by Gamma Irradiation

The stability of Compound 1 when sterilized by steam (autoclaving) was assessed. Significant degradation of Compound 1 was observed (data not shown). Gamma irradiation was then assessed as an alternative sterilization technique. Samples of Compound 1 were irradiated with 10-20 kGy or 20-30 kGy of gamma irradiation. As shown in Table 4, Compound 1 was chemically stable when gamma irradiated, with less than 0.5% degradation of Compound 1 observed by HPLC.

TABLE 4
Gamma irradiation of Compound 1

| | | HPLC | | |
|---|---|---|---|---|
| | | Assay | | |
| | | (% | | XRPD |
| Sample | Appearance | Remaining) | Average | (2-Theta) |
| 1 (non-irradi- | White powder | 98.50 | 98.48 ± 0.03 | Form A |
| ated control) | | | | |
| 2 (non-irradi- | White powder | 98.51 | | Form A |
| ated control) | | | | |
| 3 (non-irradi- | White powder | 98.48 | | Form A |
| ated control) | | | | |
| 4 (non-irradi- | White powder | 98.44 | | Form A |
| ated control) | | | | |
| 5 (non-irradi- | White powder | 98.46 | | Form A |
| ated control) | | | | |
| 6 (non-irradi- | White powder | 98.49 | | Form A |
| ated control) | | | | |
| 7 (10-20 kGy) | White powder | 98.23 | 98.23 ± 0.01 | Form A |
| 8 (10-20 kGy) | White powder | 98.24 | | Form A |
| 9 (10-20 kGy) | White powder | 98.22 | | Form A |
| 10 (20-30 kGy) | White powder | 98.17 | 98.14 ± 0.03 | Form A |
| 11 (20-30 kGy) | White powder | 98.13 | | Form A |
| 12 (20-30 kGy) | White powder | 98.11 | | Form A |

### 6.7. Example 5: Castor Oil-Based Solubilizers Outperform Other Excipients for Solubilizing Compound 1

The solubility of Compound 1 in different excipients was assessed. As shown in Table 5, the solubility of Compound 1 was generally at least an order of magnitude higher in castor-oil based solubilizers compared to other excipients. Relatively high solubility of Compound 1 with castor oil-based solubilizers was achieved over a pH range of 5-7. Advantageously, relatively high solubility of Compound 1 was observed in the castor oil-based solubilizers at pH values close to 7, which is a desirable pH for ophthalmic administration.

TABLE 5
Compound 1 solubility in different excipients

| | Compound 1 | | |
|---|---|---|---|
| | solubility | | |
| Excipient | (mg/mL) | pH | Physical form |
| 0.05% Tween ® 80 pH 7 | n/d | — | |
| 0.5% Tween ® 80 pH 7 | 0.078 | 7.1 | No change, Form A |
| 0.05% Tyloxapol pH 7 | n/d | — | |
| 0.5% Tyloxapol pH 7 | 0.034 | — | No change, Form A |
| 5% 2-Hydroxypropyl-β- | 0.18 | 5.28 | No change, Form A |
| cyclodextrin (HP-β-CD) pH 5 | | | |
| 5% HP-β-CD pH 6 | 0.07 | 6.13 | No change, Form A |
| 5% HP-β-CD pH 7 | 0.05 | 7.03 | No change, Form A |
| 2.5% HP-β-CD pH 5 | n/d | — | |
| 2.5% HP-β-CD pH 7 | n/d | — | |
| 5% (2-Hydroxypropyl)-γ- | 0.225 | 6.9 | No change, Form A |
| cyclodextrin (HP-γ-CD_{—} | | | |
| pH 7 | | | |
| 5% Polyvinylpyrrolidone | <LOQ | 6.97 | No change, Form A |
| (PVP) pH 7 | | | |
| 10% transcutol | <LOQ | 6.99 | No change, Form A |
| (2-(2-Ethoxyethoxy)ethanol) | | | |
| pH 7 | | | |
| 5% PEG400 pH 7 | 0.003 | 7.10 | No change, Form A |
| 5% Kolliphor ® EL pH 5 | 1.01 | 5.34 | No change, Form A |
| 5% Kolliphor ® EL pH 6 | 0.74 | 6.16 | No change, Form A |
| 5% Kolliphor ® EL pH 7 | 0.69 | 7.01 | No change, Form A |
| 2.5% Kolliphor ® EL pH 5 | n/d | — | |
| 2.5% Kolliphor ® EL pH 7 | n/d | — | |
| 5% Kolliphor ® RH40 | 0.4432 | 6.91 | |

n/d = not detected
<LOQ = below limit of quantification

Kolliphor® EL (polyoxyl 35 castor oil) and Kolliphor® RH40 (polyoxyl 40 hydrogenated castor oil) were selected based on the solubility data for further formulation screening studies.

### 6.8. Example 6: Compound 1 is Chemically Stable in Castor Oil-Based Solubilizers

The stability of Compound 1 in different excipients was assessed. As shown in Table 6, the stability of Compound 1 in various excipients when stored for 3 months was generally high, including in castor oil-based solubilizers, when measured by HPLC.

TABLE 6
Compound 1 stability in different excipients

| | 5° C. | | 25° C. | | 40° C. | |
|---|---|---|---|---|---|---|
| | HPLC | XRPD | HPLC | XRPD | HPLC | XRPD |
| Sample | (% Remaining) | (2-Theta) | (% Remaining) | (2-Theta) | (% Remaining) | (2-Theta) |
| 0.5% Kolliphor ® EL | 99.18 | Form | 99.18 | Form | 99.00 | Form |
| | | A | | A | | A |
| 0.5% Kolliphor ® | 99.18 | Form | 99.18 | Form | 99.17 | Form |
| RH40 | | A | | A | | A |
| 0.5% Kolliphor ® | 99.19 | Form | 99.02 | Form | 98.94 | Form |
| EL/0.5% (hydroxy- | | A | | A | | A |
| ethylcellulose) HEC | | | | | | |
| 0.5% Kolliphor ® | 99.18 | Form | 99.02 | Form | 97.89 | Form |
| EL/0.5% Carbopol ® | | A | | A | | A |
| 974P | | | | | | |
| 0.5% Kolliphor ® | 99.19 | Form | 99.18 | Form | 99.18 | Form |
| RH40/0.5% HEC | | A | | A | | A |
| 0.5% Kolliphor ® | 99.18 | Form | 99.17 | Form | 98.87 | Form |
| RH40/0.5% | | A | | A | | A |
| Carbopol ® 974P | | | | | | |
| 0.5% HEC | 99.18 | Form | 99.20 | Form | 99.17 | Form |
| | | A | | A | | A |
| 0.5% Carbopol ® | 99.19 | Form | 99.16 | Form | 99.14 | Form |
| 974P | | A | | A | | A |

Compound 1 initial purity was 99.19%

### 6.9. Example 7: Milling Increases Solubility of Compound 1

The effect of wet ball milling on Compound 1 particles size and solubility in Kolliphor® EL, Kolliphor® RH40, and tyloxopol s was assessed. Results are shown in Table 7A through 7I. Both Kolliphor® EL and Kolliphor® RH40 were found to solubilize Compound 1 better than tyloxopol. An increase in Compound 1 soluble fraction was observed with an increase in Kolliphor® RH40 concentration from 2% to 4%. Kolliphor® RH 40 appeared to be best wetting agent, with Kolliphor® EL also performing well.

TABLE 7A
Slurry of 10% Compound 1 and 0.25% tyloxopol
wet milled with 3 mm beads

| | Particle Size (μm) | | | Soluble Fraction |
|---|---|---|---|---|
| Time point | D10 | D50 | D90 | (mg/ml) |

| | | | | | |
|---|---|---|---|---|---|
| 10 | min | 1.44 | 3.52 | 8.32 | not determined |
| 3 | h | 0.683 | 2.018 | 4.74 | not determined |
| 18 | h | 0.472 | 1.036 | 3.44 | not determined |
| 24 | h | 0.442 | 0.881 | 3.05 | 0.015 |

TABLE 7B
Slurry of 10% Compound 1 and 0.5% tyloxopol
wet milled with 3 mm beads

| | Particle Size (μm) | | | Soluble Fraction |
|---|---|---|---|---|
| Time point | D10 | D50 | D90 | (mg/ml) |

| | | | | | |
|---|---|---|---|---|---|
| 10 | min | 1.219 | 3.36 | 8.31 | not determined |
| 3 | h | 0.791 | 2.191 | 4.84 | not determined |
| 18 | h | 0.492 | 1.179 | 3.78 | not determined |
| 24 | h | 0.461 | 1.002 | 3.51 | 0.076 |

TABLE 7C
Slurry of 10% Compound 1 and 1.0% Kolliphor ® EL
wet milled with 3 mm beads

| | Particle Size (μm) | | | Soluble Fraction |
|---|---|---|---|---|
| Time point | D10 | D50 | D90 | (mg/ml) |

| | | | | | |
|---|---|---|---|---|---|
| 10 | min | 1.209 | 3.24 | 7.87 | not determined |
| 3 | h | 0.716 | 2.286 | 5.88 | not determined |
| 18 | h | 0.464 | 1.195 | 3.79 | not determined |
| 24 | h | 0.418 | 0.911 | 3.5 | 0.27 |

TABLE 7D
Slurry of 10% Compound 1 and 2.0% Kolliphor ® EL
wet milled with 3 mm beads

| | Particle Size (μm) | | | Soluble Fraction |
|---|---|---|---|---|
| Time point | D10 | D50 | D90 | (mg/ml) |

| | | | | | |
|---|---|---|---|---|---|
| 10 | min | 1.37 | 3.54 | 8.95 | 0.565 |
| 3 | h | 0.693 | 1.912 | 4.12 | 0.563 |

| | | | | |
|---|---|---|---|---|
| 3 h - second run | 0.696 | 1.981 | 4.45 | 0.602 |

| | | | | | |
|---|---|---|---|---|---|
| 18 | h | 0.449 | 0.909 | 3.36 | not determined |
| 24 | h | 0.402 | 0.773 | 3.38 | 0.528 |

TABLE 7E
Slurry of 10% Compound 1 and 1.0% Kolliphor ® RH40
wet milled with 3 mm beads

| | Particle Size (μm) | | | Soluble Fraction |
|---|---|---|---|---|
| Time point | D10 | D50 | D90 | (mg/ml) |

| | | | | | |
|---|---|---|---|---|---|
| 10 | min | 1.226 | 3.13 | 7.35 | not determined |
| 3 | h | 0.548 | 1.412 | 4.16 | not determined |
| 18 | h | 0.459 | 1.092 | 4 | not determined |
| 24 | h | 0.41 | 0.821 | 3.71 | 0.344 |

TABLE 7F
Slurry of 10% Compound 1 and 2.0% Kolliphor ® RH40
wet milled with 3 mm beads

| | Particle Size (μm) | | | Soluble Fraction |
|---|---|---|---|---|
| Time point | D10 | D50 | D90 | (mg/ml) |

| | | | | | |
|---|---|---|---|---|---|
| 10 | min | 1.348 | 3.51 | 8.26 | 0.588 |
| 3 | h | 0.719 | 2.13 | 4.55 | 0.603 |
| 18 | h | 0.472 | 1.13 | 3.92 | 0.702 |
| 24 | h | 0.416 | 0.821 | 3.62 | 0.669 |

TABLE 7G
Slurry of 10% Compound 1 and 2% Kolliphor ® RH40
wet milled with 1 mm beads

| | Particle Size (μm) | | | Soluble Fraction |
|---|---|---|---|---|
| Time point | D10 | D50 | D90 | (mg/ml) |

| | | | | | |
|---|---|---|---|---|---|
| 10 | min | 1.211 | 2.914 | 6.25 | not determined |
| 3 | h | 0.511 | 1.041 | 2.823 | not determined |
| 18 | h | 0.393 | 0.729 | 2.999 | not determined |
| 24 | h | 0.38 | 0.726 | 3.62 | 0.65 |

TABLE 7H
Slurry of 10% Compound 1 and 4% Kolliphor ® RH40
wet milled with 1 mm beads

| Time | Particle Size (μm) | | | Soluble Fraction |
|---|---|---|---|---|
| point | D10 | D50 | D90 | (mg/ml) |
| 10 min | 1.133 | 2.855 | 6.24 | not determined |
| 3 h | 0.497 | 1.021 | 2.993 | not determined |
| 18 h | 0.255 | 0.526 | 2.164 | not determined |
| 24 h | 0.237 | 0.498 | 1.81 | 1.196 |

TABLE 7I
Compound 1 soluble fraction comparison with different
wetting agents/surfactants

| Surfactant/Wetting agent and Bead size | Soluble Fraction (mg/ml) |
|---|---|
| 0.25% Tyloxapol @ 24 hr, 3 mm beads | 0.015 |
| 0.5% Tyloxapol @ 24 hr, 3 mm beads | 0.076 |
| 1% Kolliphor ® EL @ 24 hr, 3 mm beads | 0.27 |
| 2% Kolliphor ® EL @ 24 hr, 3 mm beads | 0.528 |
| 1% Kolliphor ® RH-40 @ 24 hr, 3 mm beads | 0.344 |
| 2% Kolliphor ® RH-40 @ 24 hr, 3 mm beads | 0.669 |
| 2% Kolliphor ® RH40, @ 24 hr, 1 mm beads | 0.65 |
| 4% Kolliphor ® RH40 @ 24 hr, 1 mm beads | 1.196 |

### 6.10. Example 8: Viscosity of Carbomer 974P and NaCl Solutions

A study was performed to assess the viscosity of solutions of Carbomer 974P and sodium chloride at different concentrations. All viscosity measurements were performed with a Brookfield viscometer at 25° C., 10 RPM, and spindle CP-52. Results are shown in Table 8. Carbomer 974P at 0.5% w/w and 0.25% NaCl at 0.25% were selected for inclusion in Compound 1 formulations.

TABLE 8
Viscosity of Carbomer 974P and NaCl solutions

| | | Before/post |
|---|---|---|
| Vehicle | Viscosity | autoclaving |
| Carbomer 0.3% w/w solution + | 235.4 cps, | before autoclave |
| 0.1% NacL, pH 7 | 236.2 cps | |
| Carbomer 0.3% w/w solution + | 244.6 cps | post autoclaving |
| 0.1% NacL, pH 7, post | | |
| autoclaving | | |
| Carbomer 0.5% w/w solution + | 755.6 cps | before autoclave |
| 0.2% NacL, pH 7 | | |
| Carbomer 0.5% w/w solution + | 793.3 cps | post autoclaving |
| 0.2% NacL, pH 7, post | | |
| autoclaving | | |
| Carbomer 0.5% w/w solution + | 558 cps | before autoclave |
| 0.25% NacL, pH 7 | | |
| Carbomer 0.5% w/w solution + | 584 cps | post autoclaving |
| 0.25% NacL, pH 7 post | | |
| autoclaving | | |
| Carbomer 0.5% w/w solution + | 417.6 cps | before autoclave |
| 0.3% NacL, pH 7 | | |
| Carbomer 0.5% w/w solution + | 441.7 cps | post autoclaving |
| 0.3% NacL, pH 7 post | | |
| autoclaving | | |

### 6.11. Example 9: Compound 1 Formulations without HEC Exhibit Less Settling

In view of the ball milling studies and vehicle selection studies described herein, eight formulations of Compound 1 with various excipients were made. The components and initial characterization of the formulations are shown in Table 9A and Table 9B.

TABLE 9A
Compound 1 Formulations

| | Composition (values % w/w) | | | | | | | |
|---|---|---|---|---|---|---|---|---|
| Component | A | B | C | D | E | F | G | H |
| Compound 1 | 1 | 1 | 1 | 1 | 1 | 1 | 0.5 | 0.5 |
| Kolliphor ® | 0.5 | 0.5 | 0.5 | 1 | — | — | 0.5 | 0.5 |
| RH40 | | | | | | | | |
| Kolliphor ® EL | | — | — | — | 1 | 1 | | — |
| Carbopol ® | 0.5 | — | 0.5 | 0.5 | 0.5 | — | 0.5 | — |
| 974P | | | | | | | | |
| Hydroxyethyl | — | 0.9 | — | — | — | 0.9 | — | 0.9 |
| cellullose | | | | | | | | |
| 250HX | | | | | | | | |
| Sodium | 0.25 | 0.8 | 0.25 | 0.25 | 0.25 | 0.8 | 0.25 | 0.8 |
| Chloride | | | | | | | | |
| Glycerin | — | — | 1.5 | — | — | — | — | — |
| Mannitol | 3.0 | — | — | 3.0 | 3.0 | — | 3.0 | — |
| Tromethamine | q.s. to | — | q.s. to | q.s. to | q.s. to | q.s. to | q.s. to pH 7.0 | — |
| | pH 7.0 | — | pH 7.0 | pH 7.0 | pH 7.0 | pH 7.0 | | |
| HCl | q.s. to | q.s. to | q.s. to | q.s. to | q.s. to | q.s. to | q.s. to pH 7.0 | q.s. to pH 7.0 |
| | pH 7.0 | pH 7.0 | pH 7.0 | pH 7.0 | pH 7.0 | pH 7.0 | | |
| NaOH | — | q.s. to | — | — | — | — | — | q.s. to pH 7.0 |
| | | pH 7.0 | | | | | | |
| Purified water | q.s. | q.s. | q.s. | q.s. | q.s | q.s. | q.s. | q.s. |

TABLE 9B
Compound 1 Formulation Characterization

| | Z- | | Solubility | |
|---|---|---|---|---|
| | Average | | (soluble | Average drop |
| | Particle | Final | fraction) | size* |
| Formulation | size (nm) | pH | (mg/mL) | (mg) ± SD |
| A | 418.8 | 7.1 | 0.05 | 31.18 ± 0.99 |
| B | 402.8 | 7.0 | 0.06 | 30.64 ± 1.08 |
| C | 422.6 | 7.08 | 0.07 | 30.65 ± 0.51 |
| D | 412.8 | 6.97 | 0.15 | 30.59 ± 0.93 |
| E | 402.5 | 7.13 | 0.10 | 30.56 ± 0.75 |
| F | 380.5 | 6.95 | 0.15 | 29.87 ± 1.00 |
| G | 424.3 | 7.03 | 0.05 | 31.54 ± 1.48 |
| H | 419.3 | 7.08 | 0.06 | 32.26 ± 0.75 |

*8 mL Droptainer with 15 mm flat tip

All formulations had a Z-average particle size below 1 μm. The final pH and solubility (soluble fraction) was obtained as expected (target pH 7.0 and target solubility above or equal to 0.1 mg/mL). The average drop size was within a range suitable for ophthalmic use and the variability on drop size was narrow.

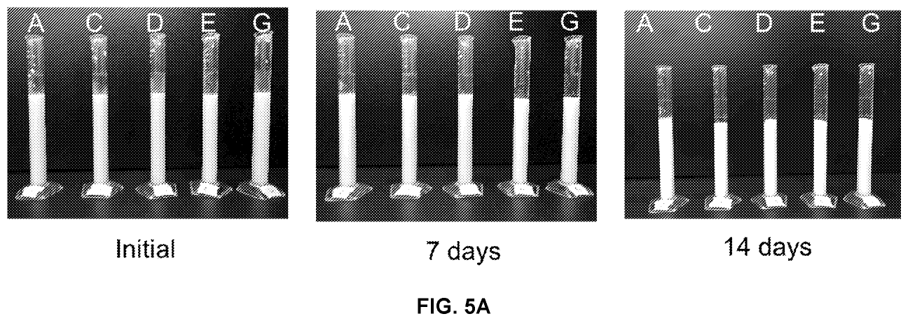

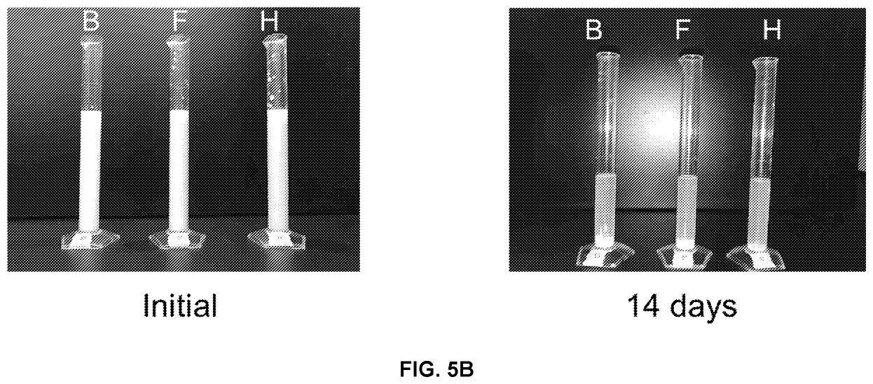

Settling of the formulations was then assessed visually at 7 and 14 days after being stored at room temperature (approximately 20° C.). As shown in FIGS. 5A-5B, the formulations that did not include HEC did not exhibit noticeable settling (FIG. 5A), while those that included HEC exhibited noticeable settling at 14 days (FIG. 5B).

### 6.12. Example 10: Compound 1 Formulations with Castor Oil-Based Solubilizers Perform Better than Comparator Formulation in PK Studies

Six Compound 1 formulations were selected for a PK study, five of which contain a vehicle with a castor oil-based solubilizer and a carbomer, and one of which includes HEC, which was previously used for a dose range finding study (data not shown). The components of the formulations are shown in Table 10.

TABLE 10
Compound 1 formulations used in PK study

| Component | 1 | | | | | |
|---|---|---|---|---|---|---|
| (% w/w) | (Control) | 2 | 3 | 4 | 5 | 6 |
| Compound 1 | 1 | 1 | 1 | 1 | 1 | 3 |
| Kolliphor ® | — | 0.5 | 0.5 | 1 | 1 | 1 |
| RH40 | | | | | | |
| Carbopol ® | — | 0.5 | 0.5 | 0.5 | 0.5 | 0.5 |
| 974P | | | | | | |
| Sodium | — | 0.25 | 0.25 | 0.25 | 0.25 | 0.25 |
| Chloride | | | | | | |
| Glycerin | — | — | 1.5 | 1.5 | 1.5 | 1.5 |
| Mannitol | — | 3.0 | — | — | — | — |
| PEG400 | 50% | — | — | — | — | — |
| HEC | 0.5% | — | — | — | — | — |
| Tromethamine | q.s. to | q.s. to | q.s. to | q.s. to | q.s. to | q.s. to |
| | pH 7.0 | pH 7.0 | pH 7.0 | pH 7.0 | pH 7.0 | pH 7.0 |
| HCl/NaOH | q.s. to | q.s. to | q.s. to | q.s. to | q.s. to | q.s. to |
| | pH 7.0 | pH 7.0 | pH 7.0 | pH 7.0 | pH 7.0 | pH 7.0 |
| Purified water | q.s. | q.s. | q.s. | q.s. | q.s. | q.s. |

Initial data on the formulations are shown in Table 11.

TABLE 11
Initial data on PK study formulations

| Formulation | Solubility (mg/mL) | Particle Size (μm) |
|---|---|---|
| 2 | 0.048 | 0.4 |
| 3 | 0.065 | 0.42 |
| 4 | 0.149 | 0.41 |
| 5 | * | 2.4 |
| 6 | * | 0.44 |

*Initial data not available

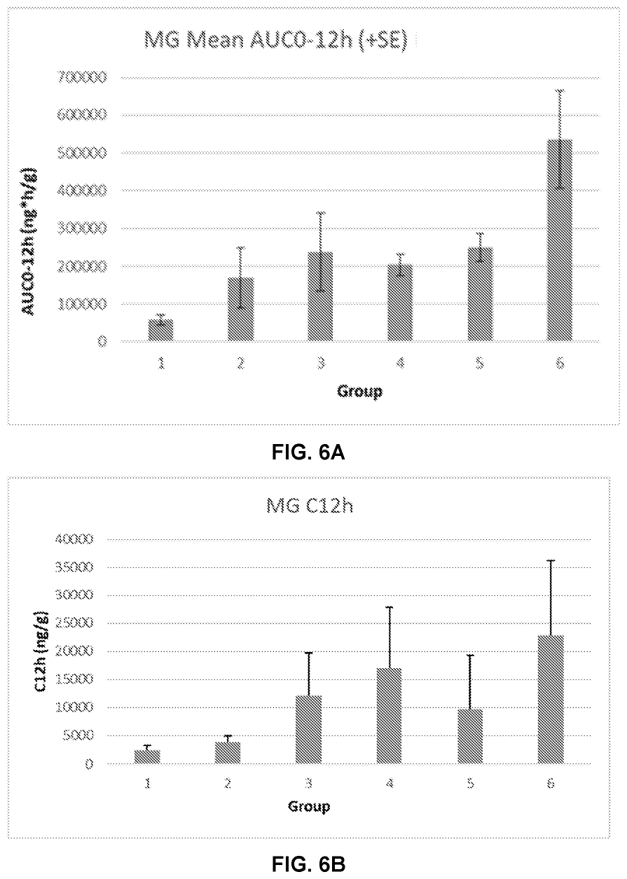

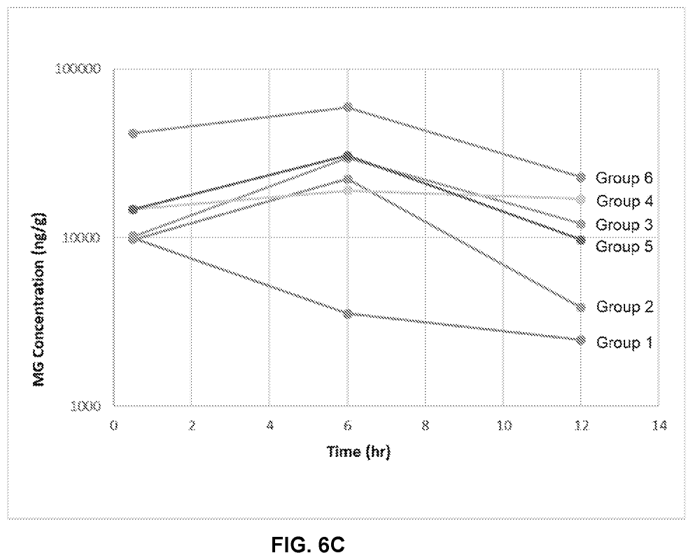

Male rabbits were assigned to 6 treatment groups, one group corresponding to each of formulations 1 to 6 (groups i to 6, respectively). An Elizabethan collar was placed on each animal and removed at two hours post dose. 30 μl of the respective formulation was administered to the top eyelid and 30 μl of the respective formulation was administered to the bottom eyelid of both eyes and spread across the entire eyelids. Meibomian gland samples were obtained at 0.5, 6 and 12 hours from the upper and lower left and right eyelids. Results are shown in FIGS. 6A-6C.

A comparison of formulations 2 and 3 allowed for the assessment of the effect of glycerin on Compound 1 exposure. A comparison of formulations 3 and 4 allowed for the assessment of the effect of increased Compound 1 soluble fraction in the formulation on Compound 1 exposure. A comparison of formulations 4 and 5 to allowed for a comparison of particle size (<1 um and <3 um, respectively) on Compound 1 exposure. A comparison of formulations 4 and 6 allowed for an assessment of different strengths (1 and 3% Compound 1, respectively).

Formulations 2-6 performed better than formulation 1 with respect to Compound 1 exposure (FIGS. 6A-6C). As shown in FIG. 6A, the rank order of the formulations when looking at mean AUC was 1<2<4<3˜ 5<6. As shown in FIG. 6B, the rank order of the formulations when looking at the 12 hour time point was 1<2<5<3<4<6. As shown in FIG. 6C, formulations 2-6 provided higher Compound 1 exposure than formulation 1 at 6 hour and 12 hour time points, and formulations 4-6 provided higher Compound 1 exposure at the 0.5 hour timepoint compared to formulations 1-3.

### 6.13. Example 11: Colloidal Stability of Compound 1 Formulations Under Temperature Cycle Stress

Four Compound 1 formulations were subjected to temperature cycle stress to assess stability. Two temperature cycles were performed. The first cycle included an 8 hour incubation at 40° C. followed by a 16 hour incubation at 4° C., which was repeated three times. The second cycle included a 6 day incubation at −20° C. followed by 1 day at 25° C. and 60% relative humidity. Results of the study are shown in Table 12

TABLE 12
Colloidal stability under temperature cycle stress

| Formulation | | Post Cycle 1 | Post Cycle 2 |
|---|---|---|---|
| A | 1% Compound 1 | D90 <3 μm | D90 <3 μm |
| | 1% Kolliphor ® RH40 | PSD remains | PSD remains |
| | 0.5% Carbopol ® 974P | colloidally stable | colloidally stable |
| | 0.25% NaCl | No crystalline | No crystalline |
| | 1.5% glycerin | form change | form change |
| B | 1% Compound 1 | D90 >20 μm | D90 >20 μm |
| | 0.5% Kolliphor ® RH40 | Significant particle | Significant particle |
| | 0.5% Carbopol ® 974P | growth in PSD | growth in PSD |
| | 0.25% NaCl | No crystalline | No crystalline |
| | 1.5% glycerin | form change | form change |
| C | 3% Compound 1 | D90 >3 μm | D90 >10 μm |
| | 1% Kolliphor ® RH40 | PSD remains | Significant particle |
| | 0.5% Carbopol ® 974P | colloidally | growth in PSD |
| | 0.25% NaCl | stable | No crystalline |
| | 1.5% glycerin | No crystalline | form change |
| | | form change | |
| D | 3% Compound 1 | D90 >10 μm | D90 >10 μm |
| | 0.5% Kolliphor ® RH40 | PSD remains | Significant particle |
| | 0.5% Carbopol ® 974P | colloidally stable | growth in PSD |
| | 0.25% NaCl | No crystalline | No crystalline |
| | 1.5% glycerin | form change | form change |

While all four formulations had D50 values below 1 μm (data not shown) after the temperature cycles, surprisingly only Formulation A had a D90 value below 3 μm after both temperature cycles, with no significant particle growth in the particle size distribution (PSD) during either temperature cycle.

### 6.14. Example 12: Compound 1 Formulation Stability Studies

Six Compound 1 formulations were made and stored at 5° C., 25° C., and 40° C. for 6 months to assess their physical and chemical stability. Degradation of Compound 1 (measured as Compound 1 associated total impurities), particle size diameter, viscosity, osmolality, pH, resuspendability, and appearance were assessed at 6 weeks (6W), 3 months (3M), and 6 months (6M). The composition of the formulations are shown in Table 13. Total impurities, particle size distribution, viscosity, osmolality, and pH values measured during the study are shown in Table 14A to Table 14E.

TABLE 13
Compound 1 formulations

| | Formulation (values % w/w) | | | | | |
|---|---|---|---|---|---|---|
| Component | 1 | 2 | 3 | 4 | 5 | 6 |
| Compound 1 | 1 | 1 | 1 | 1 | 3 | 3 |
| Kolliphor ® | 0.5 | 0.5 | 1 | 1 | 1 | 1 |
| RH40 | | | | | | |
| Carbopol ® | 0.5 | 0.5 | 0.5 | 0.5 | 0.5 | 0.5 |
| 974P | | | | | | |
| Sodium | 0.25 | 0.25 | 0.25 | 0.25 | 0.25 | 0.25 |
| Chloride | | | | | | |
| Glycerin | 0 | 1.5 | 1.5 | 1.5 | 1.5 | 1.5 |
| Mannitol | 3 | 0 | 0 | 0 | 0 | 0 |
| Tromethamine/ | adjust to | adjust to | adjust to | adjust to | adjust to | adjust to |
| Tris | pH 7.0 | pH 7.0 | pH 7.0 | pH 7.0 | pH 7.0 | pH 7.0 |
| HCl | adjust to | adjust to | adjust to | adjust to | adjust to | adjust to |
| | pH 7.0 | pH 7.0 | pH 7.0 | pH 7.0 | pH 7.0 | pH 7.0 |
| Purified Water | QS to | QS to | QS to | QS to | QS to | QS to |
| | 100% | 100% | 100% | 100% | 100% | 100% |
| Batch Size | 400 g | 400 g | 400 g | 400 g | 400 g | 420 g |

Formulation 1 and 2 were made using a wet mill slurry prepared by ball milling a mixture comprising 10% Compound 1 and 5% Kolliphor ® RH40 with 1 mm Zr beads.
Formulation 3 was made using a wet mill slurry prepared by ball milling (18 hr) a mixture comprising 10% Compound 1 and 5% Kolliphor ® RH40 with 1 mm Zr beads.
Formulation 4 was made using a wet mill slurry prepared by ball milling (3 hr) a mixture comprising 10% Compound 1 and 5% Kolliphor ® RH40 with 3 mm Zr beads.
Formulation 5 was made using a wet mill slurry prepared by ball milling (18 hr) a mixture comprising 15% Compound 1 and 5% Kolliphor ® RH40 with 1 mm Zr beads.
Formulation 6 was made using a wet mill slurry prepared by ball milling (24 hr) a mixture comprising 15% Compound 1 and 5% Kolliphor ® RH40 with 1 mm Zr beads.

TABLE 14A
Total impurities (%)

| | | 5° C. | | | 25° C. | | | 40° C. | | |
|---|---|---|---|---|---|---|---|---|---|---|
| Formulation | T0 | 6W | 3M | 6M | 6W | 3M | 6M | 6W | 3M | 6M |
| 1 | 0.83 | 0.81 | 1.12 | 1.20 | 0.84 | 1.15 | 1.17 | 0.83 | 1.21 | 1.27 |
| 2 | 0.83 | 0.79 | 1.17 | 1.14 | 0.79 | 1.18 | 1.15 | 0.86 | 1.16 | 1.29 |
| 3 | 0.84 | 0.86 | 1.16 | 1.15 | 0.86 | 1.19 | 1.16 | 0.85 | 1.18 | 1.26 |
| 4 | 0.84 | 0.80 | 1.12 | 1.16 | 0.84 | 1.15 | 1.18 | 0.86 | 1.21 | 1.24 |
| 5 | 0.83 | 0.86 | 1.14 | 1.14 | 0.83 | 1.13 | 1.17 | 0.80 | 1.18 | 1.26 |
| 6 | 0.85 | 0.83 | 1.19 | 1.15 | 0.77 | 1.17 | 1.16 | 0.82 | 1.25 | 1.24 |

TABLE 14B-1
Particle Size Distribution (μm) −5° C.

| For- | T = 0 | | | | 5° C.-6W | | | | 5° C.-3M | | | | 5° C.-6M | | | |
|---|---|---|---|---|---|---|---|---|---|---|---|---|---|---|---|---|
| mulation | MV | D10 | D50 | D90 | MV | D10 | D50 | D90 | MV | D10 | D50 | D90 | MV | D10 | D50 | D90 |
| 1 | 0.463 | 0.219 | 0.420 | 0.738 | 0.470 | 0.233 | 0.435 | 0.740 | 0.461 | 0.218 | 0.419 | 0.736 | 0.465 | 0.221 | 0.423 | 0.737 |
| 2 | 0.470 | 0.221 | 0.425 | 0.754 | 0.470 | 0.223 | 0.426 | 0.752 | 0.461 | 0.215 | 0.415 | 0.743 | 0.470 | 0.222 | 0.425 | 0.751 |
| 3 | 0.418 | 0.196 | 0.384 | 0.664 | 0.418 | 0.195 | 0.383 | 0.665 | 0.420 | 0.200 | 0.385 | 0.666 | 0.430 | 0.212 | 0.394 | 0.676 |
| 4 | 0.755 | 0.377 | 0.599 | 1.336 | 0.761 | 0.381 | 0.605 | 1.349 | 0.753 | 0.372 | 0.595 | 1.344 | 0.767 | 0.383 | 0.607 | 1.364 |
| 5 | 1.997 | 0.237 | 0.536 | 6.000 | 1.960 | 0.233 | 0.532 | 5.920 | 1.286 | 0.215 | 0.466 | 5.050 | 1.542 | 0.221 | 0.469 | 5.010 |
| 6 | 0.569 | 0.173 | 0.340 | 0.923 | 0.634 | 0.169 | 0.341 | 1.129 | 0.468 | 0.153 | 0.308 | 0.724 | 0.578 | 0.170 | 0.335 | 0.900 |

TABLE 14B-2
Particle Size Distribution (μm) −25° C.

| For- | T = 0 | | | | 25° C.-6W | | | | 25° C.-3M | | | | 25° C.-6M | | | |
|---|---|---|---|---|---|---|---|---|---|---|---|---|---|---|---|---|
| mulation | MV | D10 | D50 | D90 | MV | D10 | D50 | D90 | MV | D10 | D50 | D90 | MV | D10 | D50 | D90 |
| 1 | 0.463 | 0.219 | 0.420 | 0.738 | 0.480 | 0.234 | 0.439 | 0.757 | 0.479 | 0.230 | 0.437 | 0.760 | 0.597 | 0.238 | 0.450 | 0.781 |
| 2 | 0.470 | 0.221 | 0.425 | 0.754 | 0.492 | 0.230 | 0.439 | 0.790 | 0.499 | 0.238 | 0.445 | 0.798 | 0.522 | 0.261 | 0.465 | 0.826 |
| 3 | 0.418 | 0.196 | 0.384 | 0.664 | 0.444 | 0.217 | 0.411 | 0.695 | 0.445 | 0.216 | 0.411 | 0.699 | 0.485 | 0.266 | 0.441 | 0.737 |
| 4 | 0.755 | 0.377 | 0.599 | 1.336 | 0.784 | 0.389 | 0.618 | 1.404 | 0.803 | 0.390 | 0.622 | 1.442 | 0.828 | 0.402 | 0.640 | 1.489 |
| 5 | 1.997 | 0.237 | 0.536 | 6.000 | 3.820 | 0.337 | 2.992 | 8.450 | 3.850 | 0.341 | 3.360 | 8.460 | 3.990 | 0.343 | 3.520 | 8.750 |
| 6 | 0.569 | 0.173 | 0.340 | 0.923 | 1.143 | 0.202 | 0.417 | 3.790 | 1.208 | 0.205 | 0.425 | 3.930 | 1.267 | 0.216 | 0.445 | 4.070 |

TABLE 14B-3
Particle Size Distribution (μm) −40° C.

| For- | T = 0 | | | | 40° C.-6W | | | | 40° C.-3M | | | | 40° C.-6M | | | |
|---|---|---|---|---|---|---|---|---|---|---|---|---|---|---|---|---|
| mulation | MV | D10 | D50 | D90 | MV | D10 | D50 | D90 | MV | D10 | D50 | D90 | MV | D10 | D50 | D90 |
| 1 | 0.463 | 0.219 | 0.420 | 0.738 | 0.548 | 0.308 | 0.494 | 0.840 | 0.461 | 0.215 | 0.415 | 0.743 | 0.579 | 0.326 | 0.517 | 0.893 |
| 2 | 0.470 | 0.221 | 0.425 | 0.754 | 0.563 | 0.302 | 0.489 | 0.887 | 0.579 | 0.315 | 0.504 | 0.911 | 0.600 | 0.327 | 0.520 | 0.949 |
| 3 | 0.418 | 0.196 | 0.384 | 0.664 | 0.485 | 0.237 | 0.445 | 0.759 | 0.517 | 0.280 | 0.471 | 0.795 | 0.555 | 0.316 | 0.499 | 0.844 |
| 4 | 0.755 | 0.377 | 0.599 | 1.336 | 0.865 | 0.418 | 0.667 | 1.559 | 0.867 | 0.420 | 0.673 | 1.555 | 0.907 | 0.429 | 0.694 | 1.629 |
| 5 | 1.997 | 0.237 | 0.536 | 6.000 | 3.890 | 0.350 | 3.810 | 8.400 | 3.930 | 0.348 | 2.350 | 8.880 | 3.850 | 0.357 | 1.640 | 8.880 |
| 6 | 0.569 | 0.173 | 0.340 | 0.923 | 1.258 | 0.234 | 0.473 | 4.050 | 1.286 | 0.236 | 0.485 | 4.060 | 1.374 | 0.310 | 0.543 | 4.140 |

TABLE 14C-1
Viscosity (5° C. storage) (cPs)

| Formulation | T0 | 5° C.-6W | 5° C.-3M | 5° C.-6M |
|---|---|---|---|---|
| 1 | 278 | 226 | 258 | 267 |
| 2 | 248 | 265 | 194 | 228 |
| 3 | 225 | 268 | 232 | 249 |
| 4 | 239 | 233 | 238 | 219 |
| 5 | 264 | 240 | 268 | 190 |
| 6 | 205 | 197 | 184 | 193 |

TABLE 14C-2
Viscosity (25° C. storage) (cPs)

| Formulation | T0 | 25° C.-6W | 25° C.-3M | 25° C.-6M |
|---|---|---|---|---|
| 1 | 278 | 209 | 274 | 293 |
| 2 | 248 | 270 | 211 | 259 |
| 3 | 225 | 235 | 253 | 256 |
| 4 | 239 | 239 | 249 | 260 |
| 5 | 264 | 245 | 269 | 268 |
| 6 | 205 | 193 | 194 | 203 |

TABLE 14C-3
Viscosity (40° C. storage) (cPs)

| Formulation | T0 | 40° C.-6W | 40° C.-3M | 40° C.-6M |
|---|---|---|---|---|
| 1 | 278 | 279 | 305 | 370 |
| 2 | 248 | 286 | 318 | 329 |
| 3 | 225 | 286 | 282 | 352 |
| 4 | 239 | 240 | 292 | 339 |
| 5 | 264 | 267 | 332 | 294 |
| 6 | 205 | 220 | 254 | 307 |

TABLE 14D-1
Osmolality (5° C. storage) (mOsm/kg)

| Formulation | T0 | 5° C.-6W | 5° C.-3M | 5° C.-6M |
|---|---|---|---|---|
| 1 | 288 | 287 | 300 | 293 |
| 2 | 282 | 280 | 297 | [symbol] |
| 3 | 286 | 282 | 294 | 294 |
| 4 | 289 | 286 | 290 | 294 |
| 5 | 293 | 296 | 300 | 300 |
| 6 | 305 | 304 | 312 | 312 |

[symbol]

indicates data missing or illegible when filed

TABLE 14D-2
Osmolality (25° C. storage) (mOsm/kg)

| Formulation | T0 | 25° C.-6W | 25° C.-3M | 25° C.-6M |
|---|---|---|---|---|
| 1 | 288 | 289 | 300 | 300 |
| 2 | 282 | 281 | 301 | 294 |
| 3 | 286 | 287 | 298 | 297 |
| 4 | 289 | 285 | 296 | 300 |
| 5 | 293 | 296 | 304 | 309 |
| 6 | 305 | 305 | 317 | 319 |

TABLE 14D-3
Osmolality (40° C. storage) (mOsm/kg)

| Formulation | T0 | 40° C.-6W | 40° C.-3M | 40° C.-6M |
|---|---|---|---|---|
| 1 | 288 | 292 | 315 | 333 |
| 2 | 282 | 285 | 307 | 324 |
| 3 | 286 | 292 | 316 | 324 |
| 4 | 289 | 293 | 307 | 327 |
| 5 | 293 | 303 | 319 | 338 |
| 6 | 305 | 315 | 333 | 356 |

TABLE 14E-1
pH (5° C. storage)

| Formulation | T0 | 5° C.-6W | 5º C.-3M | 5º C.-3M |
|---|---|---|---|---|
| 1 | 7.0 | 7.0 | 7.1 | 6.9 |
| 2 | 7.0 | 7.1 | 7.1 | 7.0 |
| 3 | 7.0 | 7.0 | 7.1 | 6.9 |
| 4 | 7.0 | 7.0 | 7.0 | [symbol] |
| 5 | 7.0 | 7.0 | 7.0 | 6.9 |
| 6 | 6.9 | 7.1 | 7.0 | 6.9 |

[symbol]

indicates data missing or illegible when filed

TABLE 14E-2
pH (25° C. storage)

| Formulation | T0 | 25° C.-6W | 25° C.-3M | 25° C.-6M |
|---|---|---|---|---|
| 1 | 7.0 | 7.0 | 7.0 | 6.9 |
| 2 | 7.0 | 7.1 | 7.1 | 7.0 |
| 3 | 7.0 | 6.9 | 7.1 | 7.0 |
| 4 | 7.0 | 7.0 | 7.0 | 7.0 |
| 5 | 7.0 | 7.0 | 6.9 | 6.9 |
| 6 | 6.9 | 7.0 | 7.1 | 6.9 |

TABLE 14E-3
pH (40° C. storage)

| Formulation | T0 | 40° C.-6W | 40° C.-3M | 40° C.-6M |
|---|---|---|---|---|
| 1 | 7.0 | 7.1 | 7.0 | 6.9 |
| 2 | 7.0 | 7.1 | 7.1 | 6.9 |
| 3 | 7.0 | 7.0 | 7.1 | 6.9 |
| 4 | 7.0 | 7.0 | 6.9 | 7.0 |
| 5 | 7.0 | 6.9 | 7.0 | 7.0 |
| 6 | 6.9 | 7.0 | 7.0 | 6.9 |

As shown in Table 14A, the amount of total impurities in the formulations was 0.83-0.85 prior to storage and increased approximately 0.39-0.46% over 6 months at 40° C.

As shown in Tables 14B-1 to 14B-3, particle size distribution remained stable, with D90 values remaining below 10 μm at 6 months.

As shown in Tables 14C-1 to 14C-3, viscosity remained stable, with viscosity at 6 months generally remaining close to the initial viscosity.

As shown in Tables 14D-1 to 14D-3, osmolality remained stable, with relatively minor changes from initial values being measured at 6 months.

As shown in Tables 14E-1 to 14E-3, pH remained stable, with only minor changes from initial pH values being measured at 6 months.

Resuspendability of the formulations did not significantly change during the course of the study, as each formulation was observed to be a homogeneous suspension without lumps at each time point. Similarly, appearance of the formulations did not significantly change during the course of the study, as each formulation was white in color at each time point.

The stability studies show that the six formulations are stable when stored for up to at least six months at all storage conditions.

### 7. SPECIFIC EMBODIMENTS

While various specific embodiments have been illustrated and described, it will be appreciated that various changes can be made without departing from the spirit and scope of the invention(s). The present invention is exemplified by the numbered embodiments set forth below.

1. A formulation comprising 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof and one or more pharmaceutically acceptable carriers.

2. The formulation of embodiment 1, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is at least 0.1% w/w.

3. The formulation of embodiment 1, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is at least 0.3% w/w.

4. The formulation of embodiment 1, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is at least 0.5% w/w.

5. The formulation of embodiment 1, wherein the 3-((3-(4-(2-(isobutylsulfonyl)phenoxy)-3-(trifluorome

[symbol]

2,4-oxadiazol-5-yl)methyl)-5,5-dimethyl-1-(2-morpholinoethyl)imidazolidine-2,4-dione or a pharmaceutically acceptable salt thereof is present in the formulation in an amount that is at least 1% w/w.

6. The formulation of embodiment 1, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is at least 2% w/w.

7. The formulation of any one of embodiments 1 to 6, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is no more than 5% w/w.

8. The formulation of any one of embodiments 1 to 6, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is no more than 4% w/w.

9. The formulation of any one of embodiments 1 to 6, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is no more than 3% w/w.

10. The formulation of any one of embodiments 1 to 5, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is no more than 2% w/w.

11. The formulation of any one of embodiments 1 to 4, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is no more than 1% w/w.

12. The formulation of embodiment 1, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is 0.1% w/w.

13. The formulation of embodiment 1, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is 0.2% w/w.

14. The formulation of embodiment 1, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is 0.3% w/w.

15. The formulation of embodiment 1, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is 0.4% w/w.

16. The formulation of embodiment 1, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is 0.5% w/w.

17. The formulation of embodiment 1, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is 1% w/w.

18. The formulation of embodiment 1, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is 2% w/w.

19. The formulation of embodiment 1, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is 3% w/w.

20. The formulation of any one of embodiments 1 to 19, wherein the one or more pharmaceutically acceptable carriers comprises one or more castor oil-based solubilizers.

21. The formulation of embodiment 20, wherein the one or more castor oil-based solubilizers are present in the formulation in an amount that is at least 0.1% w/w.

22. The formulation of embodiment 20, wherein the one or more castor oil-based solubilizers are present in the formulation in an amount that is at least 0.2% w/w.

23. The formulation of embodiment 20, wherein the one or more castor oil-based solubilizers are present in the formulation in an amount that is at least 0.3% w/w.

24. The formulation of embodiment 20, wherein the one or more castor oil-based solubilizers are present in the formulation in an amount that is at least 0.4% w/w.

25. The formulation of embodiment 20, wherein the one or more castor oil-based solubilizers are present in the formulation in an amount that is at least 0.5% w/w.

26. The formulation of embodiment 20, wherein the one or more castor oil-based solubilizers are present in the formulation in an amount that is at least 1% w/w.

27. The formulation of any one of embodiments 20 to 26, wherein the one or more castor oil-based solubilizers are present in the formulation in an amount that is no more than 5% w/w.

28. The formulation of any one of embodiments 20 to 26, wherein the one or more castor oil-based solubilizers are present in the formulation in an amount that is no more than 4% w/w.

29. The formulation of any one of embodiments 20 to 26, wherein the one or more castor oil-based solubilizers are present in the formulation in an amount that is no more than 3% w/w.

30. The formulation of any one of embodiments 20 to 26, wherein the one or more castor oil-based solubilizers are present in the formulation in an amount that is no more than 2% w/w.

31. The formulation of any one of embodiments 20 to 26, wherein the one or more castor oil-based solubilizers are present in the formulation in an amount that is no more than 1% w/w.

32. The formulation of embodiment 20, wherein the one or more castor oil-based solubilizers are present in the formulation in an amount that is 0.5% w/w.

33. The formulation of embodiment 20, wherein the one or more castor oil-based solubilizers are present in the formulation in an amount that is 1% w/w.

34. The formulation of embodiment 20, wherein the one or more castor oil-based solubilizers are present in the formulation in an amount that is 5% w/w.

35. The formulation of any one of embodiments 20 to 34, wherein the one or more castor oil-based solubilizers comprise polyoxyl 40 hydrogenated castor oil and/or polyoxyl 35 castor oil.

36. The formulation of embodiment 35, wherein the one or more castor oil-based solubilizers comprise polyoxyl 40 hydrogenated castor oil.

37. The formulation of embodiment 35 or embodiment 36, wherein the one or more castor oil-based solubilizers comprise polyoxyl 35 castor oil.

38. The formulation of any one of embodiments 1 to 37, wherein the one or more pharmaceutically acceptable carriers comprise one or more carbomers.

39. The formulation of embodiment 38, wherein the one or more carbomers are present in the formulation in an amount that is at least 0.1% w/w.

40. The formulation of embodiment 38, wherein the one or more carbomers are present in the formulation in an amount that is at least 0.2% w/w.

41. The formulation of embodiment 38, wherein the one or more carbomers are present in the formulation in an amount that is at least 0.3% w/w.

42. The formulation of embodiment 38, wherein the one or more carbomers are present in the formulation in an amount that is at least 0.4% w/w.

43. The formulation of embodiment 38, wherein the one or more carbomers are present in the formulation in an amount that is at least 0.5% w/w.

44. The formulation of any one of embodiments 38 to 43, wherein the one or more carbomers are present in the formulation in an amount that is no more than 1% w/w.

45. The formulation of any one of embodiments 38 to 43, wherein the one or more carbomers are present in the formulation in an amount that is no more than 0.9% w/w.

46. The formulation of any one of embodiments 38 to 43, wherein the one or more carbomers are present in the formulation in an amount that is no more than 0.8% w/w.

47. The formulation of any one of embodiments 38 to 43, wherein the one or more carbomers are present in the formulation in an amount that is no more than 0.7% w/w.

48. The formulation of any one of embodiments 38 to 43, wherein the one or more carbomers are present in the formulation in an amount that is no more than 0.6% w/w.

49. The formulation of any one of embodiments 38 to 43, wherein the one or more carbomers are present in the formulation in an amount that is no more than 0.5% w/w.

50. The formulation of embodiment 38, wherein the one or more carbomers are present in the formulation in an amount that is 0.5% w/w.

51. The formulation of any one of embodiments 38 to 50, wherein the one or more carbomers comprise carbomer homopolymer type B.

52. The formulation of any one of embodiments 1 to 51, wherein the one or more pharmaceutically acceptable carriers comprise one or more salts.

53. The formulation of embodiment 52, wherein the one or more salts comprises sodium chloride.

54. The formulation of embodiment 53, wherein the sodium chloride is present in the formulation in an amount that is at least 0.1% w/w.

55. The formulation of embodiment 53, wherein the sodium chloride is present in the formulation in an amount that is at least 0.2% w/w.

56. The formulation of any one of embodiments 53 to 55, wherein the sodium chloride is present in the formulation in an amount that is no more than 0.5% w/w.

57. The formulation of any one of embodiments 53 to 55, wherein the sodium chloride is present in the formulation in an amount that is no more than 0.4% w/w.

58. The formulation of any one of embodiments 53 to 55, wherein the sodium chloride is present in the formulation in an amount that is no more than 0.3% w/w.

59. The formulation of embodiment 53, wherein the sodium chloride is present in the formulation in an amount that is 0.25% w/w.

60. The formulation of any one of embodiments 1 to 59, wherein the one or more pharmaceutically acceptable carriers comprise one or more tonicity enhancers.

61. The formulation of embodiment 60, wherein the one or more tonicity enhancers comprise an ionic tonicity enhancer.

62. The formulation of embodiment 61, wherein the ionic tonicity enhancer is sodium chloride.

63. The formulation of any one of embodiments 1 to 62, wherein the one or more pharmaceutically acceptable carriers comprise one or more humectants.

64. The formulation of embodiment 63, wherein the one or more humectants comprise glycerin.

65. The formulation of embodiment 64, wherein the glycerin is present in the formulation in an amount that is at least 0.5% w/w.

66. The formulation of embodiment 64, wherein the glycerin is present in the formulation in an amount that is at least 1% w/w.

67. The formulation of any one of embodiments 64 to 66, wherein the glycerin is present in the formulation in an amount that is no more than 3% w/w.

68. The formulation of any one of embodiments 64 to 66, wherein the glycerin is present in the formulation in an amount that is no more than 2% w/w.

69. The formulation of embodiment 64, wherein the glycerin is present in the formulation in an amount that is 1.5% w/w.

70. The formulation of any one of embodiments 63 to 69, wherein the one or more humectants comprise propylene glycol.

71. The formulation of any one of embodiments 63 to 70, wherein the one or more humectants comprise mannitol.

72. The formulation of embodiment 71, wherein the mannitol is present in the formulation in an amount that is at least 1% w/w.

73. The formulation of embodiment 71, wherein the mannitol is present in the formulation in an amount that is at least 2% w/w.

74. The formulation of any one of embodiments 71 to 73, wherein the mannitol is present in the formulation in an amount that is no more than 5% w/w.

75. The formulation of any one of embodiments 71 to 73, wherein the mannitol is present in the formulation in an amount that is no more than 4% w/w.

76. The formulation of embodiment 71, wherein the mannitol is present in the formulation in an amount that is 3% w/w.

77. A formulation comprising:
a) 0.1% w/w to 3% w/w 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof;
b) 0.1% w/w to 2.5% w/w polyoxyl 40 hydrogenated castor oil;
c) 0.1% w/w to 1% w/w carbomer homopolymer type B;
d) 0.1% w/w to 0.5% w/w sodium chloride;
e) 0.5% w/w to 3% w/w glycerin or 1% w/w to 5% w/w mannitol;
f) optionally, HCl and/or tomethamine and/or NaOH in quantities sufficient (qs) to provide a pH of 6.8 to 7.2; and
g) water in a quantity sufficient (qs) to 100%.

78. The formulation of embodiment 77, comprising:
a) 0.1% w/w to 3% w/w 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof;
b) 0.5% w/w to 1% w/w polyoxyl 40 hydrogenated castor oil;
c) 0.5% w/w carbomer homopolymer type B;
d) 0.25% w/w sodium chloride;
e) 1.5% w/w glycerin or 3% mannitol;
f) optionally, HCl and/or tomethamine and/or NaOh in quantities sufficient (qs) to provide a pH of 6.8 to 7.2; and
g) water in a quantity sufficient (qs) to 100%.

79. The formulation of embodiment 77 or embodiment 78, which comprises 0.1% w/w 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof.

80. The formulation of embodiment 77 or embodiment 78, which comprises 0.3% w/w 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof.

81. The formulation of embodiment 77 or embodiment 78, which comprises 1% w/w 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof.

82. The formulation of embodiment 77 or embodiment 78, which comprises 3% w/w 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof.

83. The formulation of any one of embodiments 77 to 82, which comprises 0.5% w/w polyoxyl 40 hydrogenated castor oil.

84. The formulation of any one of embodiments 77 to 82, which comprises 1% w/w polyoxyl 40 hydrogenated castor oil.

85. The formulation of any one of embodiments 77 to 84, which comprises 1.5% w/w glycerin.

86. The formulation of any one of embodiments 1 to 85, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or pharmaceutically acceptable salt thereof is radiation sterilized.

87. The formulation of embodiment 86, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or pharmaceutically acceptable salt thereof is gamma irradiated.

88. The formulation of embodiment 86, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or pharmaceutically acceptable salt thereof is X-ray irradiated.

89. The formulation of any one of embodiments 1 to 88, which comprises 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione.

90. The formulation of any one of embodiments 1 to 88, which comprises a pharmaceutically acceptable salt of 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione.

91. The formulation of any one of embodiments 1 to 89, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione is Form A.

92. The formulation of any one of embodiments 1 to 91, wherein the formulation is an ophthalmic formulation.

93. The formulation of any one of embodiments 1 to 92, wherein the formulation is a topical formulation.

94. The formulation of embodiment 93, wherein the formulation is a topical formulation for administration to skin.

95. The formulation of embodiment 94, which is a topical formulation for administration to eyelids.

96. The formulation of any one of embodiments 1 to 95, which is an aqueous suspension.

97. The formulation of embodiment 96, wherein the particles in the aqueous suspension have a D90 particle size of no more than 20 μm.

98. The formulation of embodiment 97, wherein the D90 particle size is no more than 15 μm.

99. The formulation of embodiment 97, wherein the D90 particle size is no more than 10 μm.

100. The formulation of embodiment 97, wherein the D90 particle size is no more than 5 μm.

101. The formulation of embodiment 97, wherein the D90 particle size is no more than 3 μm.

102. The formulation of embodiment 97, wherein the D90 particle size is no more than 2 μm.

103. The formulation of embodiment 97, wherein the D90 particle size is no more than 1 μm.

104. The formulation of any one of embodiments 97 to 103, wherein the D90 particle size is at least 0.5 μm.

105. The formulation of embodiment 104, wherein the D90 particle size is at least 0.6 μm.

106. The formulation of any one of embodiments 96 to 105, wherein the particles in the aqueous suspension have a D50 particle size of no more than 5 μm.

107. The formulation of embodiment 106, wherein the D50 particle size is no more than 4 μm.

108. The formulation of embodiment 106, wherein the D50 particle size is no more than 3 μm.

109. The formulation of embodiment 106, wherein the D50 particle size is no more than 2 μm.

110. The formulation of embodiment 106, wherein the D50 particle size is no more than 1 μm.

111. The formulation of embodiment 106, wherein the D50 particle size is no more than 0.9 μm.

112. The formulation of embodiment 106, wherein the D50 particle size is no more than 0.8 μm.

113. The formulation of embodiment 106, wherein the D50 particle size is no more than 0.7 μm.

114. The formulation of embodiment 106, wherein the D50 particle size is no more than 0.6 μm.

115. The formulation of embodiment 106, wherein the D50 particle size is no more than 0.5 μm.

116. The formulation of embodiment 106, wherein the D50 particle size is no more than 0.4 μm.

117. The formulation of embodiment 106, wherein the D50 particle size is no more than 0.3 μm.

118. The formulation of any one of embodiments 106 to 117, wherein the D50 particle size is at least 0.2 μm.

119. The formulation of any one of embodiments 106 to 116, wherein the D50 particle size is at least 0.3 μm.

120. The formulation of any one of embodiments 106 to 115, wherein the D50 particle size is at least 0.4 μm.

121. The formulation of any one of embodiments 106 to 114, wherein the D50 particle size is at least 0.5 μm.

122. The formulation of any one of embodiments 106 to 113, wherein the D50 particle size is at least 0.6 μm.

123. The formulation of any one of embodiments 106 to 112, wherein the D50 particle size is at least 0.7 μm.

124. The formulation of any one of embodiments 96 to 123, wherein the particles in the aqueous suspension have a D10 particle size of no more than 2 μm.

125. The formulation of embodiment 124, wherein the D10 particle size is no more than 1 μm.

126. The formulation of embodiment 124, wherein the D10 particle size is no more than 0.9 μm.

127. The formulation of embodiment 124, wherein the D10 particle size is no more than 0.8 μm.

128. The formulation of embodiment 124, wherein the D10 particle size is no more than 0.7 μm.

129. The formulation of embodiment 124, wherein the D10 particle size is no more than 0.6 μm.

130. The formulation of embodiment 124, wherein the D10 particle size is no more than 0.5 μm.

131. The formulation of embodiment 124, wherein the D10 particle size is no more than 0.4 μm.

132. The formulation of embodiment 124, wherein the D10 particle size is no more than 0.3 μm.

133. The formulation of embodiment 124, wherein the D10 particle size is v than 0.2 μm.

134. The formulation of any one of embodiments 124 to 133, wherein the D10 particle size is at least 0.1 μm.

135. The formulation of any one of embodiments 124 to 132, wherein the D10 particle size is at least 0.2 μm.

136. The formulation of any one of embodiments 124 to 131, wherein the D10 particle size is at least 0.3 μm.

137. The formulation of any one of embodiments 124 to 130, wherein the D10 particle size is at least 0.4 μm.

138. The formulation of any one of embodiments 97 to 137, wherein D90 and/or D50 and/or D10 particle size is as measured by a Microtrac S3500 particle size analyzer.

139. The formulation of any one of embodiments 97 to 137, wherein D90 and/or D50 and/or D10 particle size is as measured by a Microtrac Sync particle size analyzer.

140. The formulation of any one of embodiments 97 to 137, wherein D90 and/or D50 and/or D10 particle size is as measured by a Microtrac Bluewave particle size analyzer.

141. The formulation of any one of embodiments 1 to 140, wherein the formulation has a viscosity such that the formulation does not run off of a fingertip and/or eyelid when administered thereto.

142. The formulation of any one of embodiments 1 to 141, wherein the formulation has a viscosity such that the formulation is dispensable as a drop from a droptainer and/or a drop dispenser.

143. The formulation of embodiment 142, wherein the drop has a mass of 30 mg to 40 mg.

144. The formulation of any one of embodiments 1 to 143, wherein the viscosity of the formulation is at least 50 cPs.

145. The formulation of embodiment 144, wherein the viscosity of the formulation is at least 100 cPs.

146. The formulation of embodiment 144, wherein the viscosity of the formulation is at least 150 cPs.

147. The formulation of embodiment 144, wherein the viscosity of the formulation is at least 175 cPs.

148. The formulation of embodiment 144, wherein the viscosity of the formulation is at least 200 cPs.

149. The formulation of any one of embodiments 1 to 148, wherein the viscosity of the formulation is no more than 600 cPs.

150. The formulation of embodiment 149, wherein the viscosity of the formulation is no more than 500 cPs.

151. The formulation of embodiment 149, wherein the viscosity of the formulation is no more than 400 cPs.

152. The formulation of embodiment 149, wherein the viscosity of the formulation is no more than 350 cPs.

153. The formulation of embodiment 149, wherein the viscosity of the formulation is no more than 300 cPs.

154. The formulation of any one of embodiments 1 to 153, wherein the pH of the formulation is 6.5 to 7.5.

155. The formulation of embodiment 154, wherein the pH of the formulation is at least 6.6.

156. The formulation of embodiment 154, wherein the pH of the formulation is at least 6.7.

157. The formulation of embodiment 154, wherein the pH of the formulation is at least 6.8.

158. The formulation of embodiment 154, wherein the pH of the formulation is at least 6.9.

159. The formulation of any one of embodiments 154 to 158, wherein the pH of the formulation is no more than 7.4.

160. The formulation of any one of embodiments 154 to 158, wherein the pH of the formulation is no more than 7.3.

161. The formulation of any one of embodiments 154 to 158, wherein the pH of the formulation is no more than 7.2.

162. The formulation of any one of embodiments 154 to 158, wherein the pH of the formulation is no more than 7.1.

163. The formulation of embodiment 154, wherein the pH of the formulation is 6.9 to 7.1.

164. The formulation of embodiment 154, wherein the pH of the formulation is 7.0.

165. The formulation of any one of embodiments 1 to 164, wherein the osmolality of the formulation is at least 200 mOsm/kg.

166. The formulation of embodiment 165, wherein the osmolality of the formulation is at least 250 mOsm/kg.

167. The formulation of embodiment 165, wherein the osmolality of the formulation is at least 275 mOsm/kg.

168. The formulation of any one of embodiments 1 to 167, wherein the osmolality of the formulation is no more than 400 mOsm/kg.

169. The formulation of embodiment 168, wherein the osmolality of the formulation is no more than 375 mOsm/kg.

170. The formulation of embodiment 168, wherein the osmolality of the formulation is no more than 350 mOsm/kg.

171. The formulation of any one of embodiments 1 to 170, which forms drops having an average mass of 30 mg to 40 mg when dispensed from a 8 ml droptainer having a 15 mm flat tip.

172. The formulation of any one of embodiments 1 to 171, further comprising a preservative.

173. The formulation of any one of embodiments 1 to 171, which does not include a preservative.

174. The formulation of any one of embodiments 1 to 173, wherein upon storage of the formulation for 6 months at 5° C., 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities increase by no more than 1%.

175. The formulation of any one of embodiments 1 to 173, wherein upon storage of the formulation for 6 months at 5° C., 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities increase by no more than 0.5%.

176. The formulation of any one of embodiments 1 to 173, wherein upon storage of the formulation for 6 months at 25° C., 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities increase by no more than 1%.

177. The formulation of any one of embodiments 1 to 173, wherein upon storage of the formulation for 6 months at 25° C. 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities increase by no more than 0.5%.

178. The formulation of any one of embodiments 1 to 173, wherein upon storage of the formulation for 6 months at 40° C., 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities increase by no more than 1%.

179. The formulation of any one of embodiments 1 to 173, wherein upon storage of the formulation for 6 months at 40° C., 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities increase by no more than 0.5%.

180. The formulation of any one of embodiments 174 to 179, wherein upon the storage of the formulation for 6 months, the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione associated impurities increase by at least 0.2%, 0.3%, or 0.4%.

181. The formulation of any one of embodiments 1 to 180, wherein upon storage of the formulation for 6 months at 5° C., the D90 particle size of the formulation increases no more than 10%.

182. The formulation of any one of embodiments 1 to 180, wherein upon storage of the formulation for 6 months at 25° C., the D90 particle size of the formulation increases no more than 500%.

183. The formulation of any one of embodiments 1 to 180, wherein upon storage of the formulation for 6 months at 25° C., the D90 particle size of the formulation increases no more than 100%.

184. The formulation of any one of embodiments 1 to 180, wherein upon storage of the formulation for 6 months at 25° C., the D90 particle size of the formulation increases no more than 50%.

185. The formulation of any one of embodiments 1 to 180, wherein upon storage of the formulation for 6 months at 25° C., the D90 particle size of the formulation increases no more than 10%.

186. The formulation of any one of embodiments 1 to 180, wherein upon storage of the formulation for 6 months at 40° C., the D90 particle size of the formulation increases no more than 500%.

187. The formulation of any one of embodiments 1 to 180, wherein upon storage of the formulation for 6 months at 40° C., the D90 particle size of the formulation increases no more than 100%.

188. The formulation of any one of embodiments 1 to 180, wherein upon storage of the formulation for 6 months at 40° C., the D90 particle size of the formulation increases no more than 50%.

189. The formulation of any one of embodiments 1 to 188, wherein the viscosity of the formulation remains stable upon 6 months of storage at 5° C.

190. The formulation of any one of embodiments 1 to 189, wherein the viscosity of the formulation remains stable upon 6 months of storage at 25° C.

191. The formulation of any one of embodiments 1 to 190, wherein the viscosity of the formulation remains stable upon 6 months of storage at 40° C.

192. The formulation of any one of embodiments 1 to 191, wherein the osmolality of the formulation remains stable upon 6 months of storage at 5° C.

193. The formulation of any one of embodiments 1 to 192, wherein the osmolality of the formulation remains stable upon 6 months of storage at 25° C.

194. The formulation of any one of embodiments 1 to 193, wherein the osmolality of the formulation remains stable upon 6 months of storage at 40° C.

195. The formulation of any one of embodiments 1 to 194, wherein the pH of the formulation remains stable upon 6 months of storage at 5° C.

196. The formulation of any one of embodiments 1 to 195, wherein the pH of the formulation remains stable upon 6 months of storage at 25° C.

197. The formulation of any one of embodiments 1 to 196, wherein the pH of the formulation remains stable upon 6 months of storage at 40° C.

198. The formulation of any one of embodiments 1 to 197, wherein the resuspendability of the formulation remains stable upon 6 months of storage at 5° C.

199. The formulation of any one of embodiments 1 to 198, wherein the resuspendability of the formulation remains stable upon 6 months of storage at 25° C.

200. The formulation of any one of embodiments 1 to 199, wherein the resuspendability of the formulation remains stable upon 6 months of storage at 40° C.

201. The formulation of any one of embodiments 1 to 200, which is an aqueous suspension and which does not exhibit noticeable settling by visual inspection over a period of 7 days.

202. The formulation of any one of embodiments 1 to 201, which is an aqueous suspension and which does not exhibit noticeable settling by visual inspection over a period of 14 days.

203. The formulation of any one of embodiments 1 to 202, which does not comprise hydroxyethylcellulose (HEC).

204. A kit comprising the formulation of any one of embodiments 1 to 203 and a droptainer.

205. The kit of embodiment 204, wherein the droptainer is a 2 ml to 15 ml droptainer.

206. The kit of embodiment 205, wherein the droptainer is a 4 ml droptainer, and optionally wherein the droptainer contains 2 ml of the formulation.

207. The kit of embodiment 205, wherein the droptainer is an 8 ml droptainer, optionally wherein the droptainer contains 4 ml of the formulation.

208. The kit of any one of embodiments 204 to 207, wherein the formulation is situated within the droptainer.

209. A process of making a formulation, comprising combining a slurry comprising (a) 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoPthvnlimidiaznlidine-2,4-dione or a pharmaceutically acceptable salt thereof and one or more solubilizers, which optionally comprise one or more castor oil-based solubilizers, with (b) one or more additional pharmaceutically acceptable carriers.

210. The process of embodiment 209, further comprising a step of sterilizing 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof by radiation.

211. The process of embodiment 210, wherein the radiation is gamma radiation.

212. The process of embodiment 210, wherein the radiation is X-ray radiation.

213. The process of any one of embodiments 210 to 212, wherein the step of sterilizing 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof precedes formation of the slurry.

214. The process of any one of embodiments 209 to 213, wherein the slurry is the product of a process comprising combining 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof and one or more solubilizers to form a mixture and, subsequently, milling the mixture.

215. The process of embodiment 214, wherein the process of forming the slurring comprises milling the mixture until the D90 of particles in the mixture is less no more than 20 μm, no more than 15 μm, no more than 10 μm, no more than 5 μm, no more than 3 μm, no more than 2 μm, or no more than 1 μm.

216. The process of embodiment 214 or embodiment 215, wherein the process of forming the slurring comprises milling the mixture until the D50 of particles in the mixture is no more than no more than 5 μm, no more than 4 μm, no more than 3 μm, no more than 2 μm, or no more than 1 μm.

217. The process of any one of embodiments 214 to 216, wherein the process of forming the slurring comprises milling the mixture until the D10 of particles in the mixture is no more than no more than 2 μm or no more than 1 μm.

218. The process of any one of embodiments 214 to 217, wherein the process of forming the slurring comprises milling th

[symbol]

t least 12 hours.

219. The process of embodiment 218, wherein the process of forming the slurring comprises milling the mixture for at least 18 hours.

220. The process of embodiment 219, wherein the process of forming the slurring comprises milling the mixture for at least 24 hours.

221. The process of any one of embodiments 214 to 220, wherein the process of forming the slurring comprises milling the mixture for up to 24 hours.

222. The process of any one of embodiments 214 to 220, wherein the process of forming the slurring comprises milling the mixture for up to 36 hours.

223. The process of any one of embodiments 214 to 222, wherein the milling is ball milling.

224. The process of embodiment 223, wherein the ball milling is performed with beads having a diameter of 1 mm to 5 mm.

225. The process of embodiment 224, wherein the beads have a diameter of 1 mm.

226. The process of embodiment 224, wherein the beads have a diameter of 3 mm.

227. The process of any one of embodiments 214 to 226, further comprising a step of forming the slurry.

228. The process of any one of embodiments 209 to 227, wherein the formulation is a formulation according to any one of embodiments 1 to 203.

229. A formulation produced by the process of any one of embodiments 209 to 228.

230. A method of agonizing LXR in the Meibomian glands of a subject, comprising administering a therapeutically effective amount of the formulation according to any one of embodiments 1 to 203 to the subject.

231. A method of inducing or increasing the expression of stearoyl-CoA desaturase-1 (SCD1) in the meibomian glands of a subject, comprising administering a therapeutically effective amount of the formulation according to any one of embodiments 1 to 203 to the subject.

232. A method of increasing the ratio of desaturated lipids to saturated lipids in the eye a subject, comprising administering a therapeutically effective amount of the formulation according to any one of embodiments 1 to 203 to the subject.

233. A method of lowering a subject's meibum melting temperature and/or increasing meibum outflow from a subject's meibomian glands, comprising administering a therapeutically effective amount of the formulation according to any one of embodiments 1 to 203 to the subject.

234. A method of reducing obstruction of meibum outflow from a subject's meibomian glands, comprising administering a therapeutically effective amount of the formulation according to any one of embodiments 1 to 203 to the subject.

235. The method of any one of embodiments 230 to 234, wherein the subject has dry eye disease.

236. The method of embodiment 235, wherein the dry eye disease is evaporative dry eye disease.

237. The method of any one of embodiments 230 to embodiment 236, wherein the subject has Meibomian gland dysfunction (MGD).

238. A method for the treatment of dry eye disease in a subject in need of treatment thereof, comprising administration of a therapeutically effective amount of a formulation according to any one of embodiments 1 to 203 to the subject.

239. The method of embodiment 238, wherein the dry eye disease is evaporative dry eye disease.

240. The method of embodiment 238 or embodiment 239, wherein the dry eye disease is associated with Meibomian gland dysfunction (MGD).

241. A method for the treatment of Meibomian gland dysfunction (MGD) in a subject in need of treatment thereof, comprising administration of a therapeutically effective amount of a formulation according to any one of embodiments 1 to 203 to the subject.

242. The method of any one of embodiments 230 to 241, wherein the formulation is administered to one or more of eyelids of the subject.

243. The method of embodiment 242, wherein the formulation is administered by an applicator.

244. The method of embodiment 243, wherein the applicator is a finger.

245. A formulation according to any one of embodiments 1 to 203, for use in a method of agonizing LXR in the Meibomian glands of a subject.

246. A formulation according to any one of embodiments 1 to 203, for use in a method of inducing or increasing the expression of stearoyl-CoA desaturase-1 (SCD1) in the meibomian glands of a subject.

247. A formulation according to any one of embodiments 1 to 203, for use in a method of increasing the ratio of desaturated lipids to saturated lipids in the eye a subject.

248. A formulation according to any one of embodiments 1 to 203, for use in a method of lowering a subject's meibum melting temperature and/or increasing meibum outflow from a subject's meibomian glands.

249. A formulation according to any one of embodiments 1 to 203, for use in a method of reducing obstruction of meibum outflow from a subject's meibomian glands.

250. A formulation according to any one of embodiments 1 to 203, for use in a method of treating dry eye disease in a subject in need thereof, optionally wherein the dry eye disease is evaporative dry eye disease.

251. The formulation for use according to embodiment 250, wherein the dry eye disease is Meibomian gland dysfunction (MGD) associated dry eye.

252. A formulation according to any one of embodiments 1 to 203, for use in a method of treating Meibomian gland dysfunction (MGD) in a subject in need thereof.

253. The formulation for use according to any one of embodiments 245 to 252, wherein in the method the formulation is administered to one or more eyelids of the subject, optionally by an applicator, optionally wherein the applicator is a finger.

254. 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof, which is irradiated.

255. The 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof of embodiment 254, which is gamma irradiated.

256. The irradiated 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof of embodiment 254, which is X-ray irradiated.

257. Use of the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof according to any one of embodiments 254 to 256 in the manufacture of a medicament.

258. The use of embodiment 257, wherein the medicament is a formulation according to any one of embodiments 1 to 203.

### 8. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present specification, the teachings of the present specification are intended.

1. A formulation comprising 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof and one or more pharmaceutically acceptable carriers.

2. The formulation of claim 1, wherein the 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof is present in the formulation in an amount that is at least 0.1% w/w, at least 0.3% w/w, at least 0.5% w/w, at least 1% w/w, or at least 2% w/w, and no more than 5% w/w, no more than 4% w/w, no more than 3% w/w, no more than 2% w/w, or no more than 1% w/w.

3. The formulation of claim 1, wherein the one or more pharmaceutically acceptable carriers comprises one or more castor oil-based solubilizers, wherein the one or more castor oil-based solubilizers are present in the formulation in an amount that is at least 0.1% w/w, at least 0.2% w/w, at least 0.3% w/w, at least 0.4% w/w, at least 0.5% w/w, or at least 1% w/w, and no more than 5% w/w, no more than 4% w/w, no more than 3% w/w, no more than 2% w/w, or no more than 1% w/w.

4. The formulation of claim 3, wherein the one or more castor oil-based solubilizers comprise polyoxyl 40 hydrogenated castor oil and/or polyoxyl 35 castor oil.

5. The formulation of claim 1, wherein the one or more pharmaceutically acceptable carriers comprise one or more carbomers, wherein the one or more carbomers are present in the formulation in an amount that is at least 0.1% w/w, at least 0.2% w/w, at least 0.3% w/w, at least 0.4% w/w, or at least 0.5% w/w, and no more than 1% w/w, no more than 0.9% w/w, no more than 0.8% w/w, no more than 0.7% w/w, no more than 0.6% w/w, or no more than 0.5% w/w.

6. The formulation of claim 5, wherein the one or more carbomers comprise carbomer homopolymer type B.

7. The formulation of claim 1, wherein the one or more pharmaceutically acceptable carriers comprise one or more salts, wherein the one or more salts comprises sodium chloride, present in the formulation in an amount that is at least 0.1% w/w, or at least 0.2% w/w, and no more than 0.5% w/w, no more than 0.4% w/w, no more than 0.3% w/w, or wherein the sodium chloride is present in the formulation in an amount that is 0.25% w/w.

8. The formulation of claim 1, wherein the one or more pharmaceutically acceptable carriers comprise one or more tonicity enhancers.

9. The formulation of claim 8, wherein the one or more tonicity enhancers comprise an ionic tonicity enhancer, wherein the ionic tonicity enhancer is sodium chloride.

10. The formulation of claim 1, wherein the one or more pharmaceutically acceptable carriers comprise one or more humectants, wherein the one or more humectants comprise glycerin, wherein the glycerin is present in the formulation in an amount that is at least 0.5% w/w, or at least 1% w/w, and no more than 3% w/w, or no more than 2% w/w, or wherein the glycerin is present in the formulation in an amount that is 1.5% w/w.

11. The formulation of claim 10, wherein the one or more humectants comprise propylene glycol, or mannitol, and wherein the mannitol is present in the formulation in an amount that is at least 1% w/w, or at least 2% w/w and no more than 5% w/w, or no more than 4% w/w, or wherein the mannitol is present in the formulation in an amount that is 3% w/w.

12. A formulation comprising:
a) 0.1% w/w to 3% w/w 3-((3-(4-(2-(isobutylsulfonyl)phenoxy)-3-(trifluoromethyl)phenyl)-1,2,4-oxadiazol-5-yl)methyl)-5,5-dimethyl-1-(2-morpholinoethyl)imidazolidine-2,4-dione or a pharmaceutically acceptable salt thereof;
b) 0.1% w/w to 2.5% w/w polyoxyl 40 hydrogenated castor oil;
c) 0.1% w/w to 1% w/w carbomer homopolymer type B;
d) 0.1% w/w to 0.5% w/w sodium chloride;
e) 0.5% w/w to 3% w/w glycerin or 1% w/w to 5% w/w mannitol;
f) optionally, HCl and/or tomethamine and/or NaOH in quantities sufficient (qs) to provide a pH of 6.8 to 7.2; and
g) water in a quantity sufficient (qs) to 100%.

13. (canceled)

14. (canceled)

15. The formulation of claim 1, wherein the formulation is an ophthalmic formulation or a topical formulation for administration to skin or eyelids.

16. The formulation of claim 1, which is an aqueous suspension, wherein the particles in the aqueous suspension have a D90 particle size of no more than 20 μm, no more than 15 μm, no more than 10 μm, no more than 5 μm, no more than 3 μm, no more than 2 μm, or no more than 1 μm, and at least 0.5 μm, at least 0.6 μm.

17. The formulation of claim 1, wherein the viscosity of the formulation is at least 50 cPs, at least 100 cPs, at least 150 cPs, at least 175 cPs, or at least 200 cPs, and no more than 600 cPs, no more than 500 cPs, no more than 400 cPs, no more than 350 cPs, or no more than 300 cPs.

18. The formulation of claim 1, wherein the pH of the formulation is 6.5 to 7.5, and wherein the osmolality of the formulation is at least 200 mOsm/kg, at least 250 mOsm/kg, or at least 275 mOsm/kg, and no more than 400 mOsm/kg, no more than 375 mOsm/kg, or no more than 350 mOsm/kg.

19. The formulation of claim 1, which does not comprise hydroxyethylcellulose (HEC).

20. (canceled)

21. (canceled)

22. (canceled)

23. A method of lowering a subject's meibum melting temperature and/or increasing meibum outflow from a subject's meibomian glands, comprising administering a therapeutically effective amount of the formulation according to claim 1 to the subject.

24. A method of reducing obstruction of meibum outflow from a subject's meibomian glands, comprising administering a therapeutically effective amount of the formulation according to claim 1 to the subject.

25. A method for the treatment of dry eye disease or Meibomian gland dysfunction (MGD) in a subject in need of treatment thereof, comprising administration of a therapeutically effective amount of a formulation according to claim 1 to the subject.
